(19) United States
(12) Patent Application Publication
(10) Pub. No.: US 2022/0026426 A1
(43) Pub. Date: Jan. 27, 2022

(54) SYSTEM FOR DETERMINING PERITONITIS USING HOMODIMER NEUTROPHIL GELATINASE-ASSOCIATED LIPOCALIN

(71) Applicants: Fresenius Medical Care Holdings, Inc., Waltham, MA (US); FRESENIUS MEDICAL CARE DEUTSCHLAND GMBH, Bad Homburg (DE); FRESENIUS MEDICAL CARE R&D (SHANGHAI) CO., LTD, Shanghai (CN)

(72) Inventors: Tao Jiang, Knoxville, TN (US); YongSheng Ji, Shanghai (CN); Jesus Eduardo Muñoz-Silva, Waltham, MA (US); Fangfang Huang, Shanghai (CN); Changna Wang, Waltham, MA (US)

(21) Appl. No.: 17/384,500

(22) Filed: Jul. 23, 2021

Related U.S. Application Data
(60) Provisional application No. 63/055,823, filed on Jul. 23, 2020.

(30) Foreign Application Priority Data
May 31, 2021 (CN) 202110605930.X

(51) Int. Cl. G01N 33/543; G01N 33/68

(52) U.S. Cl. CPC G01N 33/54388; G01N 2800/26; G01N 33/6893

(57) ABSTRACT

Described are assays, associated assay kits, and associated methods for diagnosing an infection in a subject. The assay includes a first binding molecule that specifically binds an inflammatory marker (i.e., neutrophil gelatinase-associated lipocalin (NGAL) homodimer) in a sample taken from the subject, and a second binding molecule that binds a marker indicative of the presence of a pathogen in the sample. The assay and methods are for the diagnosis of peritonitis in peritoneal dialysis patients.

### CROSS-REFERENCE TO RELATED APPLICATION

This application claims the benefit under 35 U.S.C. § 119(e) of U.S. Provisional Patent Application No. 63/055,823 filed on Jul. 23, 2020, and the benefit under 35 U.S.C. § 119(a) of Chinese Application No. 202110605930.X filed May 31, 2021, the contents of each of which are incorporated herein by this reference.

### TECHNICAL FIELD

This patent application relates generally to medical devices, and more particularly to a diagnostic system, apparatus, and associated methods useful for, among other things, diagnosing and treating a peritoneal dialysis patient for peritonitis.

### BACKGROUND

Peritonitis is a major cause of morbidity and mortality in peritoneal dialysis (“PD”) patients globally. According to the International Society for Peritoneal Dialysis (“ISPD”) guidelines, peritonitis can be diagnosed when at least two of the following are present: (1) clinical features consistent with peritonitis, e.g., abdominal pain and/or cloudy dialysis effluent (“CDE”); (2) dialysis effluent with a white blood cell count (“WBC”) greater than 100/μL or greater than 0.1×10⁹/L (after a dwell time of at least two hours), with more than 50% of the WBCs being polymorphonuclear (“PMN”); and (3) a positive peritoneal dialysis effluent (“PDE”) culture.

However, in practice, when a patient presents in the clinic with symptoms of peritonitis, it typically takes clinicians from a couple of hours to a few days to get the results of a white blood cell count and PDE culture. There is also about a 10% culture-negative rate even with infections. A fast, reliable way to confirm infection is needed to prevent patient deterioration due to delay in treatment.

Once an infection diagnosis is made (without knowing the specific causative agent(s)), the nephrologist typically initiates empirical antibiotic therapy immediately. Such therapy typically includes administering at least two different broad spectrum antibiotics that collectively cover most of the gram-positive and gram-negative bacteria, along with an anti-fungal agent to prevent secondary fungal peritonitis until the causative agent(s) and drug susceptibility tests become available.

The long-term use of broad spectrum antibiotics is however known to be associated with the risk of developing drug-resistant bacteria and an increased risk of subsequent fungal infection. Therefore, an early, rapid, and culture-independent method is needed to identify the causative agent(s) of infection, support the prescription of suitable antibiotics, and reduce some of these unintended complications.

At present, a simple device is not readily available for at-home peritoneal dialysis patients or for clinicians in dialysis clinics to confirm the presence of infection as well as to identify the putative causative agents or class of causative agents.

A product from Mologic Ltd. (UK) detects certain inflammatory markers, such as IL-6 and MMP8, in PDE to alert peritoneal dialysis patients of inflammation and encourage further investigation. However, the product does not specifically identify the causative agent(s) of infection, which would assist in, e.g., prescribing antibiotics. Furthermore, inflammation may be present in some cases where there is no infection, such as inflammation caused by chemical irritants, mechanical injury to the peritoneum, or other conditions. In such cases, inflammatory status alone does not provide sufficient information for a clinician to make a timely and specific decision about treatment.

PCT International Publication WO2018/060708 describes a method of chemically detecting leukocytes (e.g., using redox indicators), and optionally simultaneously discriminating between gram-positive and gram-negative bacteria in PDE. However, the described method is not specific for causative agent(s) of infection, and requires incubation times of at least two hours, and more typically 12 hours or more. Another limitation of the method is that it may not distinguish between gram-negative infections and infection with both gram-positive and gram-negative bacteria.

Other devices may be useful to determine Gram status of bacteria in bodily fluids, but they cannot simultaneously detect inflammation, and/or they cannot indicate specific bacterial species known that may be associated with peritonitis.

A turbidity check of PDE is another technology used by the Renal Research Institute (“RRI”) and liberDi. Ronco C, Dell′Aquila R, Rodighiero MP (eds): “Peritoneal Dialysis: A Clinical Update,” Contrib. Nephrol., Basel, Karger, 2006, vol. 150, pp 187-194. The principle is to predict peritonitis by evaluating the WBC count based upon turbidity. However, there are many reasons other than just high levels of WBCs that can change the turbidity level in PDE. Furthermore, the sensitivity of a turbidity-based WBC test may be inadequate for use with many continuous ambulatory peritoneal dialysis (“CAPD”) patients.

Yet another technology from DxNow is a portable bio-imaging system with microfluidic-based consumables for prediction of peritonitis. The principle is to capture WBCs in PDE using WBC-specific antibodies and then conducting a count. This technology is intended to provide an accurate WBC count in a short time, however, it does not provide any information regarding causative agents and the device is sophisticated and relatively expensive.

Other platforms are based on PCR or other nucleic acid-based amplification/detection methodology. While these may be sensitive, they typically require bulky and expensive equipment, specialized training, and relatively long incubation periods (2+ hours), so may not be practical in many point-of-care applications where a determination is desired in minutes or less than an hour, particularly for use in the home or remote clinic setting.

Accordingly, despite a long-term need for rapid, early, and accurate detection of inflammation and specific Gram/microbial status of peritonitis in PD patients, no suitable solution has yet been identified.

The herein described system can be used to perform, from the perspective of the user, a single test with easy sample pre-treatment.

### BRIEF SUMMARY

Described is an assay for diagnosing an infection in a subject, which assay includes a first binding molecule that specifically binds an inflammatory marker (i.e., neutrophil gelatinase-associated lipocalin (“NGAL”) homodimer) in a sample taken from the subject, and, in certain embodiments, a second binding molecule that binds a marker indicative of the presence of a pathogen in the sample. The first binding molecule and the second binding molecule may be present on one test device or test strip. The assay allows for the relatively rapid diagnosis of peritonitis or a risk of peritonitis in a patient. The fast turn-around of the assay provides for early detection of peritonitis and, thus, allows for early treatment.

Also described is an assay kit that includes a binding molecule that specifically binds an antigen indicative of an inflammatory response in the peritoneum; a binding molecule that specifically binds an antigen indicative of the presence of gram-positive bacteria; and a binding molecule that specifically binds an antigen indicative of the presence of gram-negative bacteria. Such an assay kit may further include a binding molecule that specifically binds an antigen indicative of the presence of a fungus. The assay kit may further include a secondary binding molecule or molecules capable of binding to the indicated binding molecules or antigens. The secondary binding molecules may be conjugated to a label, such as a reporter or indicator, such as a fluorescent reporter or other colorable reporter, nanoparticle, reactive particle such as an enzyme, or other reporter or indicator. The binding molecules and secondary binding molecules may be, for example, antibodies and/or aptamers.

Typically, the antigen indicative of an inflammatory response in the peritoneum is NGAL dimer (or “homodimer”), and not the monomer or heterodimer, which are more generally released by epithelial cells and other tissues in response to, for example, acute kidney injury. Lipoteichoic acid (“LTA”) is an antigen indicative of the presence of gram-positive bacteria. Lipopolysaccharide (“LPS”) is an antigen indicative of the presence of gram-negative bacteria. β-glucan is an antigen indicative of the presence of a fungus.

For diagnosing peritonitis in a subject, the pathogen will typically be at least one bacterium and/or fungus.

In certain embodiments, the assay/assay kit includes a binding molecule that specifically binds an antigen indicative of the presence of a specific pathogen species. For use with a PDE, the specific pathogen will typically be Staphylococcus aureus, Pseudomonas sp., coagulase negative staphylococci, e.g., Staphylococcus epidermidis, Staphylococcus haemolyticus, Streptococcus sp., Klebsiella sp., Candida sp., Escherichia coli, vancomycin-resistant enterococci, and combinations thereof.

Described herein is the use of a molecule that specifically binds to H-NGAL in the preparation of a composition, a strip, or a kit for diagnosis, preferably a quick diagnosis of peritonitis (or for a risk of peritonitis) by detecting H-NGAL in PDE of a subject having peritonitis or at risk of developing peritonitis. In certain embodiments, the assay is configured to bind to H-NGAL and not a heterodimer of NGAL. In other certain embodiments, the assay is configured to bind to H-NGAL and not a monomer of NGAL or a heterodimer of NGAL.

Also described herein is a molecule that specifically binds to H-NGAL protein for use in the diagnosis, preferably early diagnosis of peritonitis or a risk of peritonitis by detecting H-NGAL protein in PDE of a peritoneal dialysis subject. The molecule may be used to specifically bind to H-NGAL in the assay and assay kit described herein.

Also described herein is the use of a combination of a molecule that binds specifically to H-NGAL and another molecule that binds specifically to IL-6 in the preparation of a composition, a strip, or a kit.

Also described herein is a method of diagnosing an infection in a subject using the assay kit described herein.

Typically, the assay will be incorporated into a lateral flow device, which will produce results almost instantly, e.g., on the order of minutes. In general, a lateral flow device comprises one or more panels to detect one or more antigens.

In one embodiment, multiple lateral flow devices can be integrated together to detect multiple antigens. Each lateral flow device comprising one or more panels may share a single sample reservoir.

In another embodiment, multiple lateral flow devices can be kept in the same housing but separated to detect antigens by use of individual panels.

In another embodiment, a single lateral flow device can be built in a multiplex format to detect multiple antigens using a single panel.

The described assay(s)/assay kit(s) may further include means for filtering (e.g., a filter), concentrating (e.g., centrifugation), lysing cells, and/or enriching antigen(s) from the PDE.

Sensitivity may be enhanced by concentrating the PDE, particularly for gram-positive and gram-negative antigens/markers. In certain embodiments, a syringe is used to filter and concentrate the PDE rather than a centrifuge.

The described assay(s)/assay kit(s) may further include a buffer that specifically elutes antigen(s) from the assay kit. The buffer composition may be chosen to selectively stabilize the antigen and/or antigen-antibody complex, maintain pH, maintain or disrupt structure or binding of the antigen-antibody complex.

Further described are methods of diagnosing peritonitis in a subject who is a peritoneal dialysis patient that include utilizing the herein described assay(s) or assay kit(s) to analyze the subject's PDE.

The method involves interacting the PDE of the subject with the assay in a kit as summarized above and described in detail below. The step of interacting the PDE with the assay involves a kit comprising at least one first binding molecule that binds specifically to NGAL and at least one second binding molecule that specifically binds H-NGAL and not a monomer of NGAL or a heterodimer of NGAL. In certain embodiments, the first binding molecule, the second binding molecule, or both are conjugated with a label, such as a reporter or indicator, that enables detecting H-NGAL, and thus allowing for a diagnosis for peritonitis.

Also described herein is a method for diagnosing, preferably quick diagnosis of peritonitis or a risk of peritonitis in a peritoneal dialysis subject, comprising: detecting H-NGAL protein in peritoneal dialysis effluent of the subject, where H-NGAL protein is greater than or equal to a cutoff value, indicating that the subject has peritonitis or is at a risk of peritonitis; optionally, the method further comprises detecting IL-6 protein in the peritoneal dialysis effluent, if IL-6 protein is greater than or equal to a cutoff value, indicating that the subject has peritonitis or is at a risk of peritonitis.

In certain embodiments, the method for diagnosing peritonitis involves detecting the presence of H-NGAL protein, the detection step comprising: (a) detecting H-NGAL protein in the peritoneal dialysis effluent; or (b) detecting total NGALs in the peritoneal dialysis effluent, including monomer NGAL, H-NGAL and heterodimer forms, and detecting the monomer and heterodimer forms, so as to determine the amount of H-NGAL. Preferably, a cut-off value of 500 pg/mL for H-NGAL is used, and a cut-off value of 200 pg/mL for IL-6 is used. In certain embodiments, the method of diagnosis comprises obtaining a peritoneal dialysis effluent sample, contacting a first molecule (e.g., an antibody) that specifically binds to H-NGAL protein with the sample, optionally the first molecule is immobilized on a solid support, and detecting the formed complex.

In certain other embodiments, the method comprises: obtaining a peritoneal dialysis effluent sample; contacting a first molecule, such as an antibody, that specifically binds to H-NGAL protein with the sample, optionally the first molecule is immobilized on a solid support; adding a second molecule, such as an antibody, that specifically binds to H-NGAL protein, optionally labeled, wherein the second molecule and the first molecule bind to different epitopes on H-NGAL protein; optionally, adding a substance that specifically binds to the second molecule, such as an antibody against the second molecule, optionally labeled, and detecting the formed complex.

In certain other embodiments, the method of diagnosis comprises: obtaining a peritoneal dialysis effluent sample; contacting a first antibody molecule that specifically binds to H-NGAL protein with the sample, wherein the first antibody molecule is labeled with biotin; adding a second antibody molecule that specifically binds to H-NGAL protein, wherein the second antibody molecule is labeled with colloidal gold, and the second antibody molecule and the first antibody molecule are different antibodies binding to different epitopes on H-NGAL protein; adding streptavidin, optionally, wherein the streptavidin is immobilized on a solid support, and detecting the formed complex.

Also described herein is a method for treating peritonitis in a peritoneal dialysis subject, the method comprising: (a) detecting H-NGAL protein in a peritoneal dialysis effluent of the subject; (b) determining a level of H-NGAL; (c) comparing the level of H-NGAL against a cutoff value; and (d) administering an antibiotic therapy if the level of H-NGAL exceeds the cutoff value. In certain embodiments, the cut-off value for H-NGAL may be 500 pg/mL. In certain embodiments, administering an antibiotic therapy comprises administering a drug selected from the group consisting of ertapenem, cefoxitin, doripenem, imipenem, cilastatin, meropenem, moxifloxacin, piperacillin, tazobactam, ticarcillin, clavulanate and tigecycline; or cefepime, cefotaxime, ceftazidime or ceftriaxone, together with metronidazole; or cefazolin, cefotaxime, ceftriaxone, ciprofloxacin, levofloxacin, together with metronidazole; or cefepime, ceftazidime, ciprofloxacin, levofloxacin, together with metronidazole; or gentamicin or tobramycin together with clindamycin or metronidazole.

In certain embodiments, a method of diagnosing peritonitis in a subject who is a peritoneal dialysis patient includes detecting in a PDE from the subject an antigen indicative of an inflammatory response having been launched in the peritoneum; detecting (e.g., utilizing appropriate binding molecule(s)) in the PDE an antigen indicative of the presence of gram-positive bacteria; and detecting (e.g., utilizing appropriate binding molecule(s)) in the PDE an antigen indicative of the presence of gram-negative bacteria. Preferably, results are provided in less than an hour.

In certain embodiments, the method further includes detecting (e.g., by utilizing appropriate binding molecule(s)) in the PDE an antigen indicative of the presence of a fungus.

In certain embodiments, a method of diagnosing peritonitis in a subject who is a peritoneal dialysis patient includes detecting in a PDE from the subject an antigen indicative of an inflammatory response having been launched in the peritoneum. The antigen may be the NGAL homodimer (H-NGAL).

In certain embodiments, the method further includes filtering, concentrating, and/or enriching the PDE prior to detecting antigen(s). In certain embodiments, enrichment may be by, for example, placing 50 mL of PDE into a conical tube, followed by centrifugation of the PDE sample, and treatment with, e.g., a lysis buffer, extraction buffer, and neutralization buffer. Enrichment of a PDE sample for antigens may also be by use of an ultrafiltration (“UF”) membrane (e.g., molecular weight cutoff 29,000). In another embodiment, a syringe is used to pass/push the PDE through a UF membrane instead of a centrifuge.

Such diagnostic methods can be used (or incorporated into methods) to treat peritonitis in the subject by first diagnosing the subject as having peritonitis and then administering an appropriate antibiotic to the subject to treat the peritonitis in view of the diagnosis.

In a particularly preferred embodiment, a paper device that utilizes lateral flow assay technology and/or dry chemistry test technology is disclosed to simultaneously detect the presence of cell wall components and/or surface antigen(s) from contaminating microorganisms and/or inflammatory biomarkers in a patient's PDE. In some embodiments, lateral flow technology and/or dry chemistry test technology is used to rapidly assist clinicians in diagnosing an infection in the peritoneal dialysis patient when the patient presents in the clinic with cloudy PDE and/or other symptoms of peritonitis. In certain embodiments, detection is based upon a lateral flow test (also known as a lateral flow immunochromatographic assay), which is a simple, paper-based device used to detect the presence of a target analyte in a liquid sample without the need for specialized and costly equipment. The device aids in the determination of the contaminating microorganism(s) so that, for example, appropriate antibiotics therapy can be promptly prescribed.

The device and associated methods for using it assist clinicians (and patients and caregivers) in quickly determining the existence of an infection in a peritoneal dialysis patient and aid in providing treatment therefore so as to improve the management of peritoneal dialysis-related peritonitis. Described are assays and methods for use and in aid of the rapid detection the presence of molecular markers that are released from the patient's host immune system and/or pathogens as soon as the invasion of pathogens is thought to have occurred in the patient.

In certain embodiments, described is a diagnostic composition (e.g., a lateral flow assay, an appropriately configured paper strip, or kit for diagnosis) comprising a molecule that specifically binds to H-NGAL. Such a diagnostic composition may be used to, for example, diagnose peritonitis or risk of peritonitis in a peritoneal dialysis subject by contacting the diagnostic composition with peritoneal dialysis effluent of the peritoneal dialysis subject.

### BRIEF DESCRIPTION OF THE DRAWINGS

FIGS. 1 through 5 and 7 depict various embodiments of lateral flow devices as described herein.

FIG. 6 is a flow diagram of a process according to the disclosure.

FIG. 8 is a reference colorimetric card for use alongside an assay according to the disclosure.

FIG. 9 is an illustration of the different pads that comprise a single assay strip that may be used in a channel of a lateral flow device, as described herein.

FIG. 10 is an illustration of one embodiment according to the disclosure.

FIG. 11 is an illustration of another embodiment according to the disclosure.

FIGS. 12-14 are illustrations of assays according to the disclosure.

### DETAILED DESCRIPTION

As used herein, a “binding molecule,” e.g., an antibody (monoclonal or polyclonal) or antigen-binding fragment thereof, aptamer, affimer (peptide aptamer), receptor binding domain, or designed ankyrin repeat proteins (DARPins) is a molecule that specifically binds an antigen. The term “antigen-binding fragments” means a portion of an intact binding molecule, such as an antibody. Examples of antibody fragments include Fab, Fab′, F(ab′)2 and Fv fragments, CDR, antigen-binding site, heavy or light chain variable region, diabodies, triabodies single chain antibody molecules (“scFv”) and multi-specific antibodies formed from at least two intact antibodies or fragments thereof or peptides or polypeptides that contain at least a fragment of an immunoglobulin that is sufficient to confer antigen binding to the peptide or polypeptide, etc. An antigen-binding fragment may comprise a peptide or polypeptide comprising an amino acid sequence of at least 2, 5, 10, 15, 20, 25, 30, 35, 40, 50, 60, 70, 80, 90, 100, 125, 150, 175, 200, or 250 contiguous amino acid residues of the amino acid sequence of the antibody. The antigen-binding fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, edited by E. Harlow and D. Lane (1988), Cold Spring Harbor Laboratory, Cold Spring Harbor, N.Y. An antibody or antigen-binding fragment thereof has one or more binding sites. If there is more than one binding site, the binding sites may be identical to one another or they may be different. The term “specifically binding,” or “specifically recognize,” as used herein, in reference to the interaction of an antibody and its binding partner, e.g., an antigen, means that the interaction is dependent upon the presence of a particular amino acid sequence or structure, e.g., an antigenic determinant or epitope, on the binding partner. In other words, the antibody preferentially binds or recognizes the binding partner even when the binding partner is present in a mixture of other molecules or organisms. The binding may be mediated by covalent or noncovalent interactions or a combination of both. In yet other words, the term “specifically binding” or “specifically recognizes” means that the antibody is specifically immunoreactive with an antigenic determinant or epitope and is not immunoreactive with other antigenic determinants or epitopes. An antibody that specifically or immunospecifically binds to an antigen may bind to other peptides or polypeptides with lower affinity as determined by, e.g., radioimmunoassays (“RIA”), enzyme-linked immunosorbent assays (“ELISA”), BIACORE, or other assays known in the art. Antibodies or fragments thereof that specifically bind to an antigen may be cross-reactive with related antigens, carrying the same epitope. Preferably, antibodies or fragments thereof that specifically bind to an antigen do not cross-react with other antigens.

The strength or affinity of a specific binding interaction can be represented by the dissociation equilibrium constant (K_{D}) of the interaction. The term “K_{D}” refers to the dissociation equilibrium constant of a specific antibody-antigen interaction, which describes the binding affinity between the antibody and the antigen. The smaller the dissociation equilibrium constant, the tighter the antibody-antigen binding, and the higher the affinity between the antibody and the antigen. In certain embodiments, an antibody that specifically binds to a certain antigen (or an antibody specific to a certain antigen) means that the antibody binds to the antigen with a K_{D} of less than about 10⁻⁸ M, for example, less than about 10⁻⁸ M, 10⁻⁹ M, 10⁻¹⁰ M, 10⁻¹¹ M or even less. In certain embodiments, when K_{D}≤10×10⁻⁸ M, the antibody or antigen-binding fragment thereof of the invention is considered to specifically bind to H-NGAL or IL-6 protein. Those skilled in the art can obtain antibodies specific for H-NGAL protein, NGAL protein and IL-6 protein.

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, spatially relative terms, such as “beneath,” “below,” “lower,” “bottom,” “above,” “upper,” “top,” “front,” “rear,” “left,” “right,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, if materials in the figures are inverted, elements described as “below” or “beneath” or “under” or “on bottom of” other elements or features would then be oriented “above” or “on top of” the other elements or features. Thus, the term “above” can encompass both an orientation of above and below, depending on the context in which the term is used, which will be evident to one of ordinary skill in the art. The materials may be otherwise oriented (e.g., rotated 90 degrees, inverted, and/or flipped) and the spatially relative descriptors used herein interpreted accordingly.

As used herein, the term “configured” refers to a size, shape, material composition, and arrangement of one or more of at least one structure and at least one apparatus facilitating operation of one or more of the structure and the apparatus in a pre-determined way.

Analytes/associated specific binding molecules: In certain embodiments, the device is configured to detect one, two, or more inflammatory markers, including NGAL homodimer. The device may optionally not contain a biomarker corresponding to an antigen, such as a pathogen, in the same device.

In certain embodiments, the device is configured to detect one, two, or more inflammatory markers including NGAL homodimer (H-NGAL), as well as one, two, three, or more cell wall components, such as lipoteichoic acid (“LTA”), lipopolysaccharide (“LPS”), and β-glucan in one device.

As disclosed in WO 20200249228 (Aug. 6, 2020) and U.S. Ser. No. 16/752,518 for Rapid Diagnosis of Peritonitis in Peritoneal Dialysis Patients, filed Jan. 24, 2020, the contents of the entirety of which are incorporated herein by this reference, NGAL (neutrophil gelatinase-associated lipocalin, lipocalin 2, siderocalin, or neutrophil lipocalin) is a 25 kDa glycosylated single chain monomer and member of the lipocalin family of proteins that bind and transport small lipophilic molecules. NGAL is released by activated neutrophils, as well as renal tubule epithelial cells, cardiomyocytes, uterus, prostate, salivary glands, and other tissues, and can be found in both serum and urine. NGAL can form dimers, forming a 45 kDa disulfide-linked homodimer, small amounts of higher oligomers, and a 135 kDa heterodimer with matrix metalloproteinase 9 (“MMP 9,” gelatinase B). NGAL is involved in innate immunity. The homodimer form of NGAL is specifically released by neutrophils that infiltrate into the peritoneum immediately after the invasion of a pathogen, while the monomer and heterodimer are more generally released by tubular cells and the other indicated tissues in response, for example, to acute kidney injury. During the process of peritonitis, the level of NGAL homodimer in PDE correlates well with the quantity of neutrophils, and NGAL homodimer levels can be used to predict infection even before any clinical symptoms are noticed. With a lateral flow assay, detection of NGAL appears to be of a higher sensitivity than MMP 9 in the PDE samples. The assays described in the incorporated WO 20200249228 (Aug. 6, 2020) and U.S. Ser. No. 16/752,518 are not specific for NGAL homodimer, but also detect NGAL monomer and NGAL heterodimer.

Because the monomer and homodimer of NGAL indicate different physiological conditions, an immunoassay for diagnosing peritonitis that generally tests for NGAL without differentiating between the forms may be over inclusive. The assay described herein however has reduced false positives. Therefore, when using the NGAL homodimer as a marker for peritonitis, using a binding molecule that distinguishes between the homodimer and heterodimer forms of NGAL aids in an accurate diagnosis. Thus, in certain embodiments, the assay comprises at least one molecule that specifically binds to the NGAL homodimer and not a NGAL heterodimer. In other certain embodiments, the assay comprises at least one molecule that specifically binds to the NGAL homodimer and not a NGAL homodimer and not the NGAL heterodimer.

An IgG1 monoclonal antibody that specifically binds the homodimer form of NGAL, but not the heterodimer form is available under the trade designation “N457,” available from HyTest Ltd. of Turku, Finland (Cat. #4NG7-N457). Other antibodies available from HyTest described as having similar binding specificities as N457 include N308 and N432.

Further antibodies that detect NGAL homodimer only include ABS 038-14-02 from Invitrogen (ThermoFisher Scientific, Inc., Waltham, Mass., US) (e.g., using ABS 038-14-ABS 038-14B as the antibody pair, the assay is specific to homodimer) and 254530 from Abbiotech, Inc., of Escondido, Calif., US.

Antibodies that bind the monomer, homodimer, and heterodimer forms of NGAL include N316, N417, N422, and N461 from HyTest Ltd. Antibodies that bind the monomer, homodimer, and heterodimer forms of NGAL include N316, N417, N422, and N461 from HyTest Ltd.

An enzyme immunoassay (“sandwich immunoassay”) for detection of solely the homodimer form of NGAL containing N316 as the NGAL capture antibody and N457 as the detection antibody is referenced in “Neutrophil gelatinase-associated lipocalin (NGAL)” TechNotes, p. 2 (January 2020 by HyTest Ltd.), the contents of which are incorporated herein by this reference. While not being bound by theory, it is thought that within the context of that particular assay, binding of the NGAL molecule(s) to, e.g., the N316 capture binding molecule blocks the monomer binding site of the NGAL molecule(s) from the, e.g., N457 detection antibody, thus enabling the assay to distinguish between homodimer and the other forms. Thus, in certain embodiments, the detection antibody for NGAL in an assay may be selected from the group consisting of N457, N308, N432, and combinations of any thereof, while the capture antibody is selected from the group consisting of N316, N417, N422, N461, and combinations of any thereof.

Thus, increased levels of NGAL homodimer in the PDE indicate both the activation of innate immunity and that there is a host defense in the peritoneum, which are initiated at the beginning of peritonitis.

In certain embodiments, other inflammatory markers are also tested with the assay. Such other inflammatory markers include IL-6, TNFα, fibrinogen, MPO, HAS, fMLP, A1AT, TIMP2, TIMP1, sICAM, MMP 9, HNE, cystatin C, IL-lb, IL-8, calprotectin, RBP4, MMP 8, MMP 2, desmosine, and SPD. Antibodies that specifically bind these other inflammatory markers are described in the incorporated WO 20200249228.

In certain described embodiments, the device may also use only a gram-positive inflammatory marker(s) with an additional technology. For example, in certain embodiments, using turbidity detection technology (e.g., that of RRI) in combination with a binding molecule that binds a marker indicative of the presence of a pathogen could obviate the use of a binding molecule for an inflammatory marker.

Thus, in certain embodiments, described is a method of treating peritonitis in a subject who is a peritoneal dialysis patient, the method comprising: first determining if turbidity is present in a PDE, analyzing the subject's PDE to determine the identity of an infectious microorganism (e.g., Gram (+), Gram (−), and/or fungal infection), and then administering an appropriate antibiotic to the subject to treat the peritonitis in view of the determination of the identity of the microorganism.

In certain embodiments, turbidity detection may be used in combination with a binding molecule that binds a marker indicative of the presence of a pathogen and a binding molecule that binds an inflammatory marker. Such a combination of indicators can provide additional benefits to the patient or clinician, particularly in early diagnosis of infection and early characterization of specific agents of infection.

Peptidoglycan is a key component of the cell wall of bacteria and thus serves as a reliable marker of bacterial infection, distinguishing other causes of inflammation and/or infection.

Lipoteichoic acid (“LTA”) is a glycerol phosphate surface polymer component of the envelope of gram-positive bacteria. LTA is anchored via its glycolipids to the membrane and carries a polysaccharide chain extending into the peptidoglycan layer of the cell wall. LTA is released spontaneously into the culture medium during growth of gram-positive bacteria. LTA functions as an immune activator with characteristics very similar to lipopolysaccharide (LPS) from gram-negative bacteria. LTA binds to CD14 and triggers activation predominantly via Toll-like receptor 2. Although LTA is internalized and traffics to the Golgi, cellular activation in response to LTA occurs at the cell surface.

Antibodies that specifically bind to LTA are readily commercially available. For example, a mouse monoclonal antibody is available under Catalog #MA1-40134 from ThermoFisher Scientific. MA1-40134 detects LTA from gram-positive bacterial samples, and has been successfully used in Western blot, ELISA, flow cytometry, immunofluorescence, and immunohistochemistry (frozen) applications. Another mouse monoclonal antibody (IgG3) is available under Catalog #GWB-9E3242 from Genway Biotech, Inc. (San Diego, Calif., US). Other mouse monoclonal LTA antibodies are available, for example, under Catalog #LS-C102921, LS-C102920, LS-C202488, and LS-C757317 from LifeSpan Biosciences, Inc. (Seattle, Wash., US).

LPS is a cell wall component of gram-negative bacteria. Antibodies that specifically bind LPS are readily commercially available. For example, a rabbit polyclonal antibody preparation is available under Product No. PAB526Ge01 from Cloud-Clone Corp. that has the ability to recognize LPS in immunohistochemical staining and western blotting. A lipopolysaccharide (LPS) mouse monoclonal antibody is available under Catalog No. CAU29364 from Biomatik (Cambridge, Ontario, CA).

β-glucan is a cell wall component of fungi. Antibodies that specifically bind β-glucan are readily commercially available. See, also, U.S. Pat. No. 7,893,219 to Cassone et al. (Feb. 22, 2011), the contents of which are incorporated herein by this reference.

LTA, LPS, and β-glucan are cell wall components of gram-positive bacteria, gram-negative bacteria, and fungi, respectively. The presence of such cell wall component(s) in the patient's PDE is indicative both that infection is occurring and the specific type of causative agent(s).

In certain embodiments, other specific antigens can be detected to indicate the presence of the specific pathogen (e.g., bacteria) species in the PDE. Commonly, such specific pathogens are selected from the group consisting of Staphylococcus aureus, Pseudomonas spec., Staphylococcus epidermidis, Staphylococcus haemolyticus, vancomycin-resistant enterococci, Candida sp., Escherichia coli, and mixtures thereof.

For example, lysostaphin could be used to detect Staphylococcus aureus and exotoxin A could be used to detect Pseudomonas spec. Monoclonal and polyclonal anti-lysostaphin antibodies are commercially available from Antibody Research Corporation (St. Peters, Mo., US) under the SKUs 111145 and 111135, respectively. Polyclonal anti-Pseudomonas Exotoxin A antibodies produced in rabbit are available from Sigma-Aldrich/Millipore SiGMa under the MDL number MFCD00162779. Similarly, Staph. aureus-specific peptidoglycan can be used to specifically detect Staph. aureus in PDE. Monoclonal antibodies are available from QED Biosciences, Inc. (San Diego, Calif., US) under the Catalog Nos. 15702, 15703, and 15704; from Meridian Life Science, Inc. (Memphis, Tenn., US) under the Catalog No. C55570M, and from Abcam, Inc. (Cambridge, Mass., US) under the Product name ab37644. These antibodies recognize peptidoglycan of Staph. aureus, Protein A-negative Staph. aureus, and Staph. epidermidis.

Lateral Flow Devices:

A lateral flow assay (“LFA”) (also known as a lateral flow test (“LFT”), lateral flow device (“LFD”), lateral flow immunoassay (“LFIA”), lateral flow immunochromatographic assays, “Dipstick,” Pen-side test, Quick/Rapid test, or test strip) is a simple to use diagnostic device to confirm the presence or absence of target analytes, such as pathogens or biomarkers in humans or animals.

As used herein, a “sample” to be tested is applied to the “sample pad,” and the sample pad can be prepared according to any suitable method known in the art.

As used herein, the “conjugate pad” comprises a capture molecule, optionally labeled, that binds to the molecule to be detected in a sample.

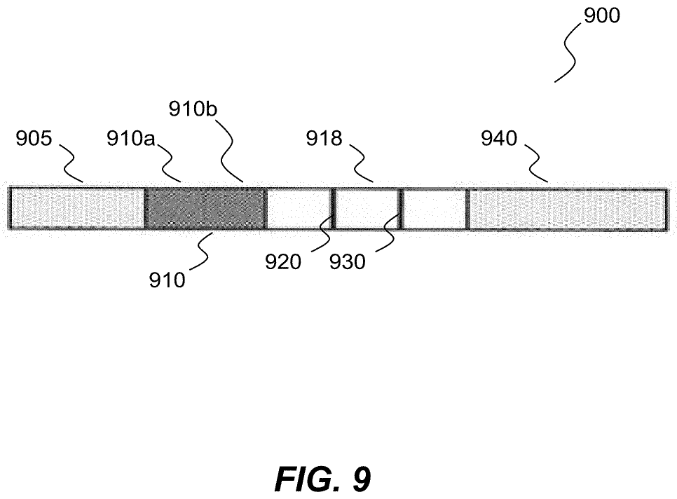

As used herein, the “biotinylated pad” comprises a biotinylated molecule that specifically binds to the molecule to be detected in a sample. As used herein, the gold-labeled pad comprises a colloidal gold-labeled molecule such as an antibody that specifically binds to the molecule to be detected or a complex formed by the molecule to be detected and a capture molecule. Examples include a biotinylated pad 910a or gold-labeled pad 910b, as illustrated in FIG. 9.

As used herein, the “nitrocellulose membrane” may comprise one or more test lines that contain immobilized detection molecules that bind to a complex formed by the molecule to be detected and a capture molecule. There is a distance between two test lines, e.g., a distance of 0.5-5 mm, such as 0.5, 1.0, 1.5, 2.0, 2.5, 3.0, 3.5, 4.0, 4.5, or 5.0 mm, so as to minimize or eliminate interference between (or among) detection results at the test lines. Setting test lines in a strip is known in the art and not discussed at length herein.

As used herein, a “label” is any molecule that can be used to label H-NGAL and/or IL-6 protein, including but not limited to, biotin, fluorescence, radioactivity, luminescence, chemistry, enzyme and particulate labels such as latex particles, colloidal gold, etc. In certain embodiments, the label is selected from the group consisting of colloidal gold and biotin.

In certain embodiments, the lateral flow assay for diagnosing peritonitis in a patient or subject comprises: a solid support having a first end and a second end, the first end comprising a sample pad, a conjugate pad, a nitrocellulose (NC) membrane, and an absorbent pad. In certain embodiments, the NC membrane comprises a control line and a test line.

In certain embodiments the conjugate pad may further comprise at least two conjugate pads. By non-limiting example, in certain embodiments, the conjugate pad 910 may comprise a biotinylated pad 910a and a gold-labeled pad 910b. The biotinylated pad 910a may comprise a biotin-labeled molecule that specifically binds to an antigen (e.g., H-NGAL, NGAL monomer, NGAL/MM9 complex, LTA, β-glucan, LPS, and IL-6). The gold-labeled pad 910b may comprise a colloidal gold-labeled second molecule that specifically binds to the antigen.

In certain embodiments, the lateral flow assay has a control line to confirm that the test is working properly, along with one or more target or test lines. The control line may comprise an immobilized substance that specifically binds to the first molecule, the second molecule, the third molecule, and/or the fourth molecule. The test line may comprise an immobilized substance that detects a complex formed by H-NGAL and the first molecule and/or the second molecule. In certain other embodiments, the test line may also comprise an immobilized substance that detects a complex formed by IL-6 and the third molecule and/or the fourth molecule. The assays are designed to incorporate intuitive user protocols and require minimal training to operate. They can be qualitative and read visually, or quantitative when combined with reader technology, such as ADxLR5®.

The control line may comprise an immobilized substance that specifically binds to the first molecule, the second molecule, the third molecule, and/or the fourth molecule. Optionally, the first molecule may be labeled.

In certain embodiments, the test line comprises a first molecule that specifically binds to H-NGAL. The first molecule may comprise a first antigen-binding fragment specific for H-NGAL. In certain embodiments, the first antigen binding fragment may be an antibody that specifically binds to an epitope of H-NGAL. In other certain embodiments, the first antigen binding fragment may bind to NGAL, either as the monomer, homodimer (H-NGAL), heterodimer form complexed with MMP-9, or a combination thereof.

In alternate embodiments, the test line comprises a second molecule that binds to a complex formed by H-NGAL and the first molecule. In other alternate embodiments, the test line comprises an immobilized substance that binds to a complex formed by H-NGAL, the first molecule, and the second molecule.

In certain embodiments, the test line comprises a second molecule that specifically binds to NGAL. The second molecule may be a second antigen binding fragment, such as an antibody, that binds to an epitope of H-NGAL, where the epitope that interacts with the second molecule is different from the epitope that interacts with the first molecule. The second molecule may exhibit a color that allows for qualitative detection when the assay is used as a point-of-care test (POCT). The second molecule capable of exhibiting a color for use in an assay is known in the art, such as horseradish peroxidase (HRP), alkaline phosphatase (AP), glucose oxidase, β-D-galactosidase, among others. In other certain embodiments, the second molecule may convert substrates into colored substrates for detection, the substrates comprising o-phenylenediamine (OPD), tetramethylbenzidine (TMB), p-nitrophenylphosphate (pNPP), diaminobenzidine (DAB), or others as is known in the art.

In certain embodiments, the first molecule and second molecule may be a first antibody molecule and a second antibody molecule, respectively. The first antibody molecule may selectively bind to a first epitope of H-NGAL in a sample, and the second antibody molecule may selectively bind to a second epitope of H-NGAL in the sample, where the first epitope and second epitope are different. The second antibody molecule may be labeled with colloidal gold. In other certain embodiments, the first antibody molecule and second antibody molecule are independently selected from N316 and N457.

In certain other embodiments, the test line may comprise a third molecule that specifically binds to an inflammatory marker, such as IL-6, TNFα, fibrinogen, MPO, HAS, fMLP, A1AT, TIMP2, TIMP1, sICAM, MMP 9, HNE, cystatin C, IL-lb, IL-8, calprotectin, RBP4, MMP 8, MMP 2, desmosine, and SPD. In certain embodiments, the third molecule specifically binds to IL-6.

In certain other embodiments, the test line may comprise a fourth molecule that specifically binds to another antigen other than H-NGAL, e.g., an inflammatory marker. In certain embodiments, the other antigen is an inflammatory marker, such as IL-6, and the fourth molecule specifically binds to IL-6. The assay may further comprise another test line for detecting a complex comprising the third molecule complexed to IL-6 or a complex comprising the third molecule and the fourth molecule complexed to IL-6. In other certain embodiments, the third and fourth molecules may be antibodies specific for IL-6, including polyclonal and monoclonal antibodies and fragments thereof. Such antibodies include M-BE8 (EP0430193; Klein, B. et al., 1991, Murine anti-interleukin 6 monoclonal antibody therapy for a patient with plasma cell leukemia, Blood, 78, 1198-1204) or M-23C7, or an antibody that binds to an epitope of the inflammatory marker.

In one embodiment, described is a lateral flow assay comprising: a solid support having a first end and a second end, the first end comprising a sample pad, a biotinylated pad, a gold-labeled pad, a nitrocellulose (NC) membrane, and an absorbent pad. In certain embodiments, the NC membrane comprises a control line and a test line. In certain other embodiments, the NC membrane may further comprise another test line, the other test line configured to detect a target molecule different from the test line.

The biotinylated pad comprises a biotinylated first molecule that specifically binds to H-NGAL. The gold-labeled pad comprises a colloidal gold-labeled second molecule that specifically binds to H-NGAL. The test line comprises immobilized streptavidin, the streptavidin capable of binding to the biotinylated first molecule complexed to H-NGAL. The control line comprises an immobilized substance that specifically binds to the first molecule and/or the second molecule. Optionally, the first molecule and the second molecule are different.

In certain embodiments, the test line and the control line are separated to enable spatial resolution of the two lines. The test line and control line may be separated by about 0.5, 1.0, 1.5, 2.0, 2.5, 3.0, 3.5, 4.0, 4.5, or 5.0 mm.

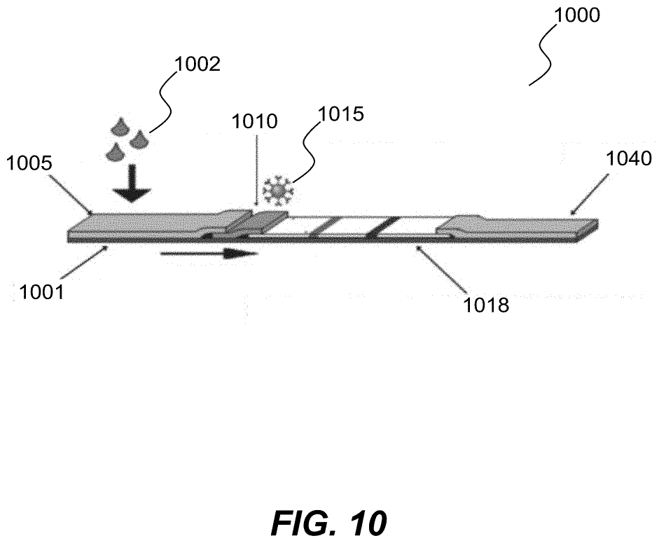

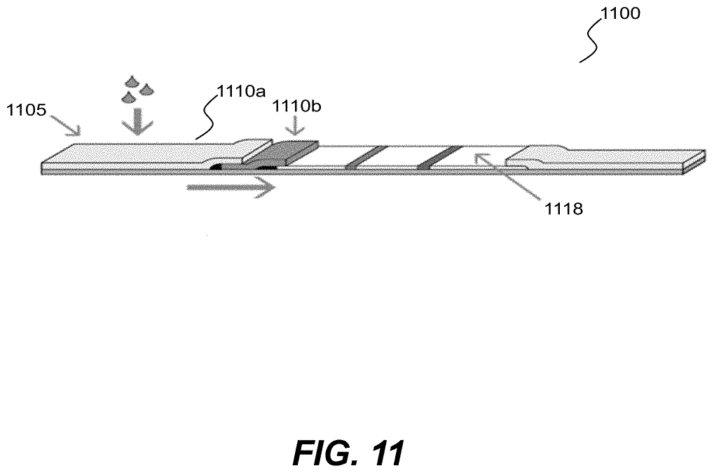

In certain embodiments, fabrication of the assay may result in portions of the assay overlapping with each other, as illustrated in FIGS. 10 and 11. As illustrated in FIG. 10, an assay 1000 receives a sample containing an analyte 1002 onto the sample pad 1005, which overlies a polyvinyl chloride (PVC) baseplate 1001. The sample pad 1005 may overlap with the conjugate pad 1010 that extends from a first end of the sample pad 1005 to an opposite end of an absorbent pad 1040. The sample pad 1005 is raised above the conjugate pad 1010, which may comprise a first molecule 1015 that specifically binds to an antigen in the analyte 1002. In certain embodiments, the conjugate pad 1010 overlaps with the NC membrane 1018, such that the conjugate pad 1010 is raised above the NC membrane 1018. In certain embodiments, the NC membrane 1018 overlaps with the absorbent pad 1040, such that the absorbent pad is raised above the NC membrane 1018. In other certain embodiments, the conjugate pad comprises a biotinylated pad 1110a and a gold-labeled pad 1110b, as illustrated in FIG. 11. The biotinylated pad overlaps 1110a with the gold-labeled pad 1110b, such that the biotinylated pad 1110a is raised above the gold-labeled pad 1110b. In yet other certain embodiments, the gold-labeled pad 1110b overlaps with the NC membrane 1118, such that the gold-labeled pad 1110b is raised above the NC membrane 1118.

Lateral flow tests are widely used in human health for point-of-care testing. They can be performed by a healthcare professional or by the patient, and in a range of settings including the laboratory, clinic, or home.

Lateral flow assays can be developed to be used in a dipstick format or in a housed cassette. Both dipsticks and housed tests will work in a similar way, dependent on the industry, sample matrix, and the market requirement.

Although a “competitive assay” could be adapted for use herein (e.g., by utilization of an enrichment method such as centrifugation to increase the sensitivity of detection), sandwich (or “non-competitive”) assays are typically preferred for use herein. In a non-competitive immunoassay format, a positive test is typically represented by the presence of a colored line at the test line position. A non-competitive immunoassay format is ideal for large molecular weight analytes with multiple antigenic sites. Generally, non-competitive immunoassays have a lower limit of detection (heightened analytical sensitivity) compared to a competitive format. Typically, non-competitive immunoassays can detect on the order of picograms/mL (parts per trillion) in comparison to nanograms/mL (parts per billion) for the competitive assay format.

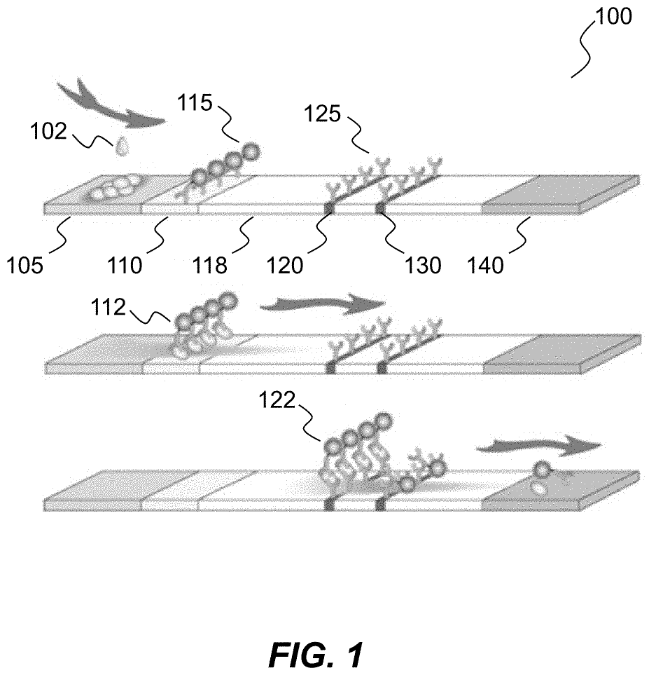

Lateral flow devices typically utilize immunoassay technology with a nitrocellulose membrane, colored latex nanoparticles (or labels such as magnetic beads or colored styrene balls), and antibodies, for quick analysis (see, e.g., FIG. 1).

When a sample from a PDE is added to the lateral flow device, the sample flows along the test device passing through a conjugate pad into a nitrocellulose membrane and then onto an absorbent pad to absorb excess sample.

The sample pad acts as the first stage of the absorption process, and in some cases contains a filter to ensure the accurate and controlled flow of the sample. The conjugate pad, which stores the conjugated labels and antibodies, receives the sample. If the target is present, the immobilized conjugated antibodies and labels bind to the target and continue to migrate along the test.

As the sample moves along the device, the binding reagents situated on the nitrocellulose membrane bind to the target at the test line. A colored line forms and the density of the line varies depending on the quantity of the target present. Some targets may require quantification to determine the target concentration such as by using a reader.

If the assay is based on colloidal gold immunochromatography, a specific antibody (or an antigen) is immobilized on the membrane in strip form, and a colloidal gold-labeled reagent (antibody) is adsorbed on the conjugate pad. When the sample to be tested is added to the sample pad at one end of the test strip, it moves forward via capillary action, dissolves the colloidal gold-labeled reagent on the conjugate pad and reacts with each other. When moving to the immobilized antibody (or antigen) area, the complex formed by the substance to be tested and the gold-labeled reagent specifically binds thereto and thus is intercepted, gathering on the test line, and the coloration result can be observed visually. Structure of a colloidal gold immunochromatography strip is schematically described in FIG. 10.

Multiplexed lateral flow assays, related methods, and devices are disclosed in U.S. 20050250141A1 to Lambert et al., (Nov. 10, 2005), the contents of which are incorporated herein by this reference, which assays are capable of simultaneously detecting multiple analytes. See also U.S. Pat. No. 6,924,153 B1 to Boehringer et al. (Aug. 2, 2005), U.S. Pat. No. 8,128,871 B2 to Petruno et al. (Mar. 6, 2012), and U.S. Pat. No. 7,785,899 B2 to Saul et al. (Feb. 18, 2005), the contents of each of which are incorporated herein by this reference, for various lateral flow test kits capable of detecting multiple analytes.

Conjugate labels used with lateral flow devices include: colloidal gold, cellulose nanobeads, fluorescent latex, paramagnetic particles, up-converting phosphors, and various fluorescent labels.

In certain embodiments, detection may be by way of using a second molecule that displays a visible or colorimetric reaction. The second molecule may be a detection antibody specific for an epitope of H-NGAL, where the capture antibody and detection antibody bind to different epitopes. In certain embodiments, the second molecule may be conjugated with horseradish peroxidase, alkaline phosphatase, glucose oxidase, β-D-galactosidase, or any combination thereof. The use of conjugated molecules is known in the art, and thus not discussed at length. In other certain embodiments, substrates may be added to the assay, the substrates comprising o-phenylenediamine (OPD), tetramethylbenzidine (TMB), p-nitrophenylphosphate (pNPP), diaminobenzidine (DAB), or combinations thereof.

In certain embodiments, the cell wall components are detected by dry chemistry if, for example, the specificity of the antibodies in the lateral flow test is inadequate. Dry chemistry is based upon specific recognition of cell wall component(s) by, e.g., enzymes.

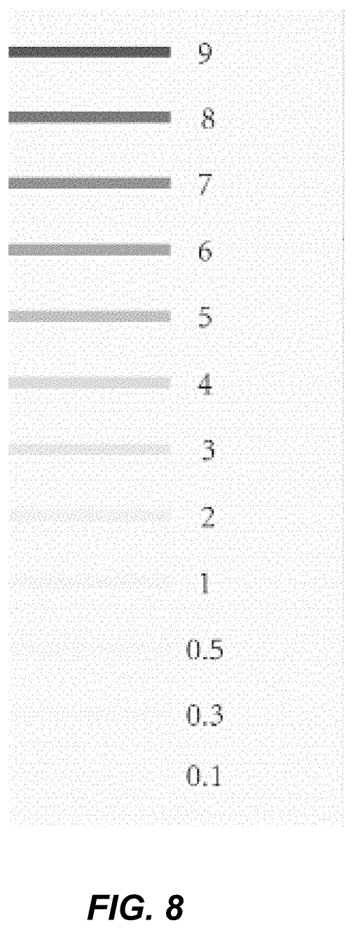

Alternatively, a more sophisticated system can be used to acquire quantitative data instead of qualitative data. For example, the immunofluorescence technology together with a reader may be utilized to measure the quantity of markers in PDE to provide more information to the patients and clinicians by comparing the obtained values with a predetermined cutoff value, as described below and illustrated in FIG. 8.

The detection of each biomarker or cell wall component is preferably carried out in an individual channel, although all detections are made simultaneously by using the same PDE sample. Such a design can prevent cross-reaction between biomarkers or cell wall component(s).

It is preferred to add PDE sample to each channel. But alternatively, more than one channel can be connected to each other to avoid repeatedly adding the PDE sample. Accordingly, described is a point-of-care device and assay for testing for peritonitis in dialysis effluent, especially in PDE, providing several advantages over conventional assays and improving peritonitis management. The device and methods are inexpensive, rapid, and easy to use by a patient or clinician with only limited training. Also described is a device and method for such testing that will rapidly allow identification of specific microbial agents of peritonitis or other infection, decreasing the time needed to confirm a peritonitis diagnosis, to facilitate early determinations of an optimal treatment regimen (e.g., specific antibiotic therapy or mixed therapy), with the possibility to replace culture testing and hospital-based laboratories altogether, where both sensitivity and specificity of testing are equivalent to laboratory methods.

A particular advantage is that the disclosed device and assay combines both inflammatory marker testing and specific infection agent testing in a single, inexpensive, and simple to use device. The device and assay gives the patient and clinician substantially more information about actual infection and appropriate peritonitis management than previous technology. For example, tests looking only at inflammatory status rely exclusively on non-specific markers of infection. Even if the test is positive for one or more inflammatory markers (alone), the clinician does not gain significant information about which specific type of therapy (e.g., specific type of antibiotic) to prescribe. The disclosed assay adds an agent-specific test (e.g., Gram status, specific bacterium, fungus, etc.) to the inflammatory marker, greatly increasing reliability of the diagnosis of pathogen-associated peritonitis, while reducing the time needed to implement the most appropriate therapy. Further, PDE samples according to the disclosed assay and device do not necessarily require pre-treatment or any special processing before they are applied to the device, so patients can perform the entire test in their homes immediately. As a result, rather than prescribing, for example, an empiric, broad-spectrum antibiotic in response to a generalized confirmation of peritonitis, the clinician can quickly prescribe a targeted antibiotic that is most suitable for treating a known gram-positive or gram-negative bacterial infection. The combined device is also cheaper and easier to use and interpret than two devices or multiple assays, and results can be read in less than 10 minutes in most cases.

In this way, patients can be treated sooner with a much higher probability of being administered with, for example, the most suitable antibiotic for their infection. In some settings, a patient in a home setting could accordingly test their own PDE, identify a specific positive result and report to their clinician, and be directed to begin a course of targeted treatment immediately. Such therapy could be initiated even where other classical signs of infection (e.g., pain, fever, cloudy effluent) are not yet present. Further, the severity and duration of peritonitis in, for example, PD patients may be reduced, allowing them to stay on PD longer without the need for more invasive forms of dialysis, such as hemodialysis. Also reduced is the use of improper antibiotics (i.e., empiric, broad-spectrum antibiotics where a more targeted therapy is indicated), thus reducing the incidence of antibiotic-resistant bacteria in the population.

In certain embodiments, a picture is taken of the results of the test, which are transmitted to a health care practitioner. In certain embodiments, a mobile telephone “app” is used to analyze the results, and provide the patient with a notation to, for example, “Call your health care provider” or “Peritonitis Confirmed.” In certain embodiments, this can further include analysis of a picture of the analyte, using technology as described in International Application No.: PCT/EP2017/000803, published on Jan. 11, 2018, the contents of which are incorporated herein by this reference.

FIGS. 1 through 5 and 7 illustrate various embodiments of a lateral flow device as described. In the depicted cases, the device is configured to detect NGAL, LTA, β-glucan, LPS, and IL-6 in one test.

As depicted in FIG. 1, some embodiments of the assay 100 comprise a volume of analyte 102 (e.g., PDE) that is deposited into a well portion of the strip, such as a sample pad 105. The PDE flows away from the well (e.g., by capillary action), down the strip, and interacts with a conjugate of appropriate labeled antibodies associated with the strip, such as at a conjugate pad 110. The appropriate antigen specifically binds to a first molecule 115 on the conjugate pad 110 to form a complex 112. Other antibodies having the same specificity as the labeled antibodies are affixed to the strip (at the solid phase), such as a test line 120. If the appropriate antigen is present in the PDE (e.g., NGAL, LTA, β-glucan, LPS, or IL-6), it will bind with the labeled antibodies and form a sandwich complex 122 at the portion of the strip 118 where the solid phase antibodies are present, and the sandwich complex 122 will be detectible. The portion of the strip may be a NC membrane 118, which may comprise a test line 120 and a control line 130. The portion at which a sandwich complex 122 may form may be the test line 120. A control line 130 is further depicted to ensure the point-of-care test (POCT), such as the assay 100, is performing properly. Excess reagent passes to the end of the strip, such as to an absorbent pad 140.

In certain embodiments, the test line 120 comprises a second molecule 125 immobilized to the solid phase. The second molecule 125 specifically binds to the complex 112. The control line 130 typically binds to the first molecule from the conjugate pad 110.

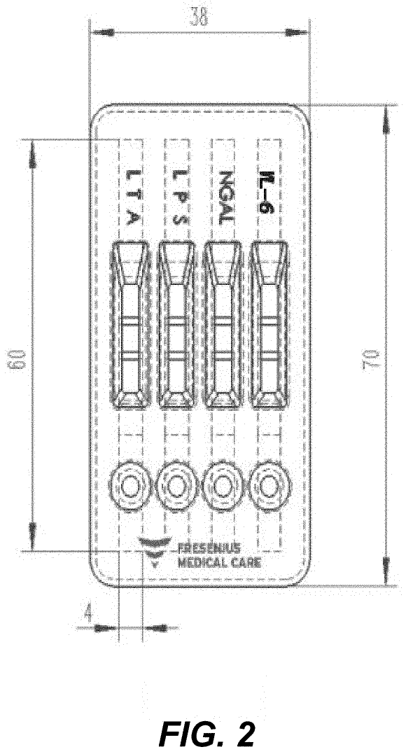

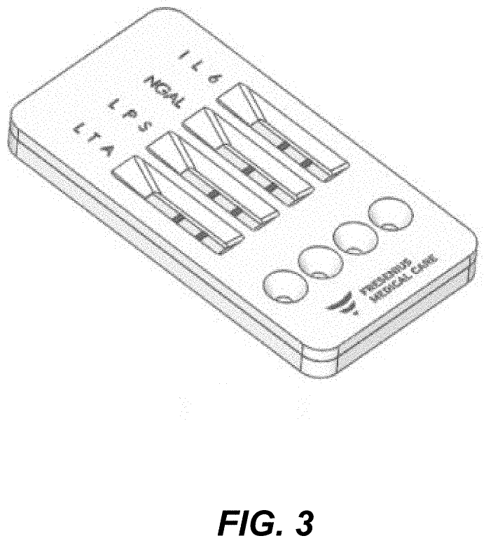

FIGS. 2 and 3 represent various configurations of a lateral flow device according to the disclosure (and FIG. 1), including four sample wells and four roughly-parallel and adjacent channels configured to detect LTA, LPS, NGAL, and IL-6, such as assays 100 as illustrated in FIG. 1. This embodiment has the advantage of taking up relatively little space, while requiring application of four relatively low-volume PDE samples to utilize all four channels. In FIGS. 2 and 3, the control lines are distal from the wells, and the test lines are closer to the wells.

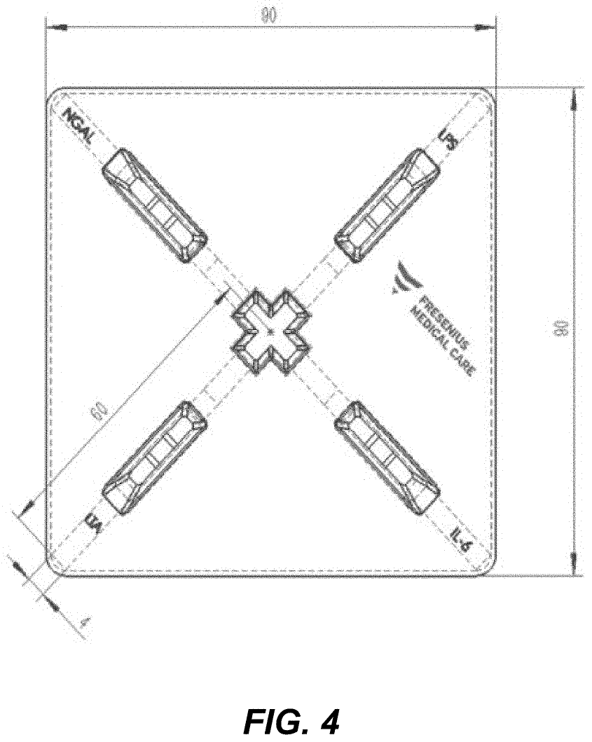

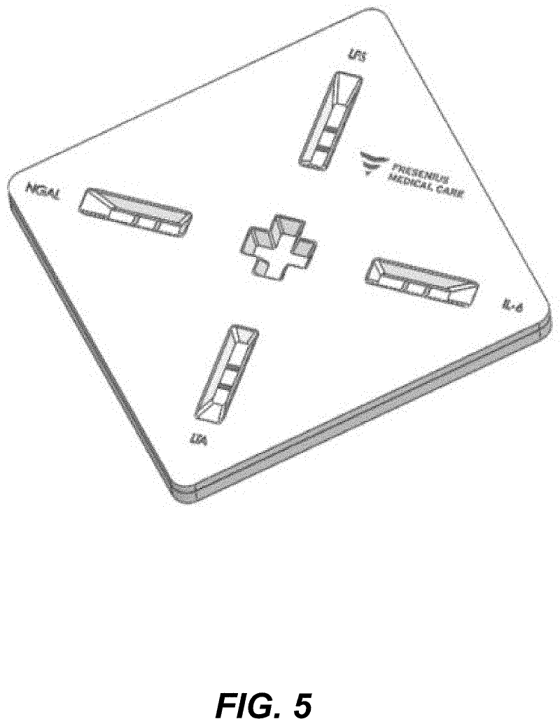

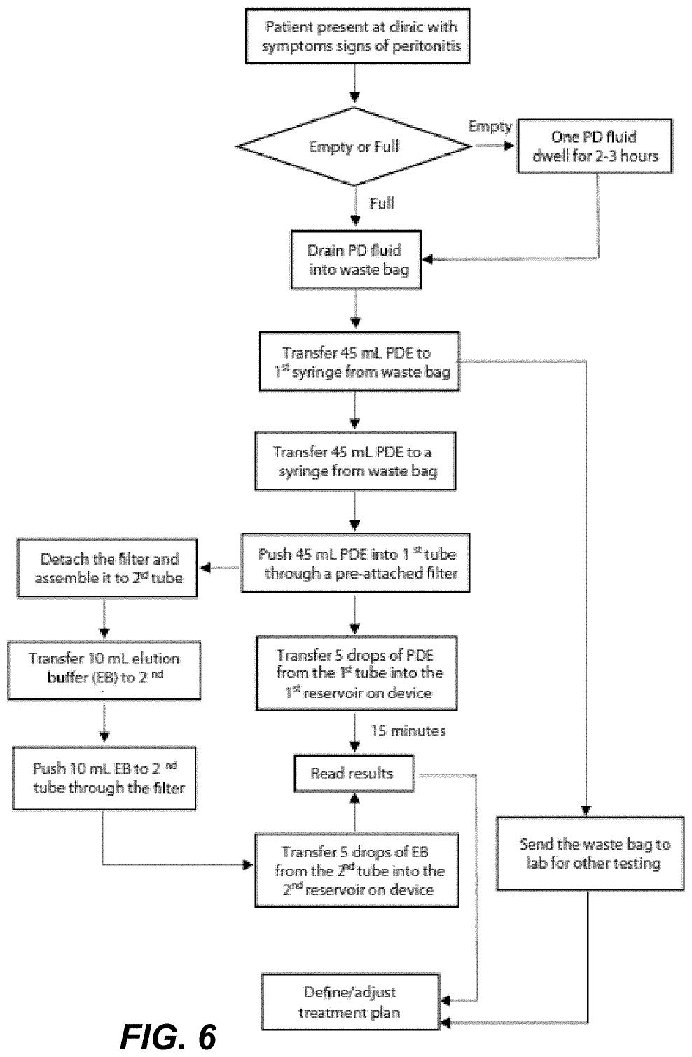

FIGS. 4 and 5 represent an alternative configuration of a lateral flow device of the disclosure, including a single, central sample well, such as 105 as illustrated in FIG. 1, and four channels that radiate away from the central well, such as 105 as illustrated in FIG. 1, configured again to detect LTA, LPS, NGAL, and IL-6, such as the assays 100 as illustrated in FIG. 1. This embodiment has the advantage of requiring only one sample of PDE to be applied by the patient or clinician.

FIGS. 2 and 4 also indicate possible dimensions (in millimeters) for the lateral flow device and ranges for such dimensions, according to certain embodiments. For example, the device depicted in FIG. 2 can have a width 38 with a Range of 30˜60 mm and a Tolerance of −1˜+1, a height 70 having a Range of 55˜100 mm and a Tolerance of −1˜+1, depicted distance 60 having a Range of 50˜80 mm and a Tolerance of −1˜+1, and depicted width 4 of the channel having a Range of 2.5-8 mm and a Tolerance of −1˜+1. The device depicted in FIG. 4 can have, e.g., a height and width 90 with a Range of 70˜120 mm and a Tolerance of −1˜+1, depicted distance 60 having a Range of 50˜80 mm and a Tolerance of −1˜+1, and depicted width of the channel having a Range of 2.5-8 mm and a Tolerance of −1˜+1.

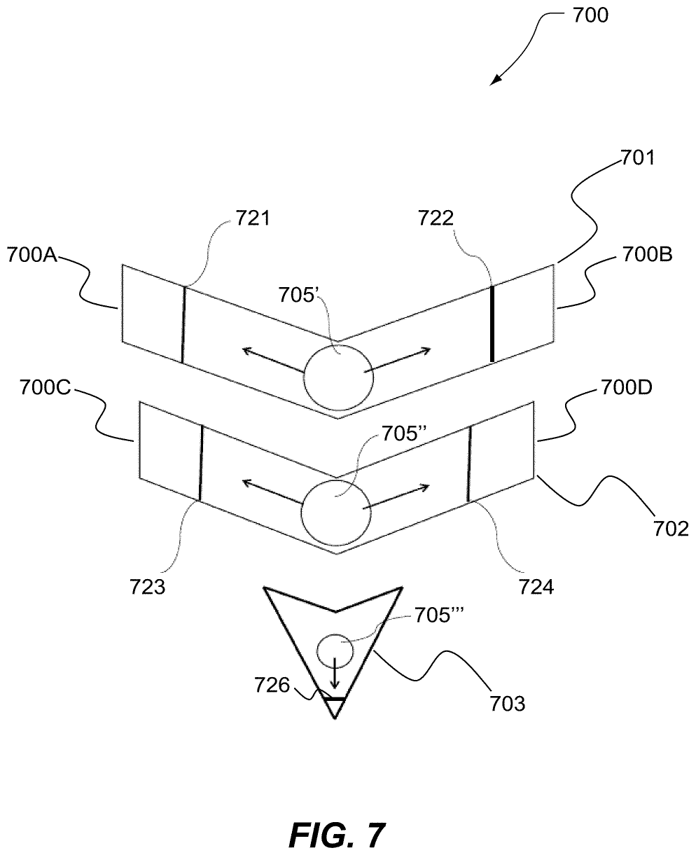

FIG. 7 illustrates an assay 700 having triple-chevron configuration (e.g., FRESENIUS™ logo), to which a sample is applied to the center 705′, 705″, 705′″ of each chevron. Each chevron comprises at least two channels, where the channels 700A, 700B, 700C, 700D may be the assays 100 as illustrated in FIG. 1 or assays 900, 1000, 1100 as illustrated in FIGS. 9-11. The sample then flows laterally or longitudinally along multiple channels to test lines where appropriate and binding reagents bind antigens that may be present in the samples. The individual chevron components comprise a first chevron 701, a second chevron 702, and a third chevron 703. Each of the chevrons 701, 702, and 703 have two channels (channels 700A, 700B, 700C, 700D . . . 700 N), wherein the channels may comprise a test line that specifically binds to an antigen at test lines 721, 722, 723, 724. In certain embodiments, test line 721 is configured to detect H-NGAL. In certain other embodiments, test line 722 comprises an immobilized molecule that specifically binds to an antigen, such as IL-6, complexed to a third molecule. In yet certain other embodiments, test line 723 is configured to detect LTA, and test line 724 is configured to detect LPS. In yet certain other embodiments, the third chevron 703 comprises a test line 726 for the detection of β-glucan. In some embodiments, the different antigens of interest are tested for at the various test lines 721, 722, 723, 724, and 726 to enable a health care provider to effectively triage the nature of renal damage the patient experiences.

As illustrated in FIG. 9, certain embodiments of the assay 900 may comprise a sample pad 905, a conjugate pad 910, a nitrocellulose membrane 918, and an absorbent pad 940. The conjugate pad 910 may further comprise sub-pads 910a, 910b . . . 910n. In certain embodiments, pad 910a is a biotinylated pad 910a. In other certain embodiments, pad 910b is a gold-labeled pad 910b. In certain embodiments, NC membrane 918 comprises a test line 920 and a control line 930. When a biotinylated pad 910a is used, the test line 920 may comprise immobilized streptavidin. An analyte containing an antigen, such as H-NGAL, flows from the sample pad 905 to the biotinylated pad 910a, where a biotinylated first molecule specifically binds to H-NGAL to produce a complex of H-NGAL/biotinylated first molecule. In certain embodiments, the gold-labeled pad comprises a molecule that specifically binds to H-NGAL in the analyte to produce a complex of H-NGAL/gold-labeled first molecule. As the complex flows through the assay 900, the test line 920 comprises an immobilized molecule that captures the H-NGAL/biotinylated first molecule complex and/or the H-NGAL/gold-labeled first molecule complex.

In certain embodiments, the test line and the control line are separated to enable spatial resolution of the two lines. The test line and control line may be separated by about 0.5, 1.0, 1.5, 2.0, 2.5, 3.0, 3.5, 4.0, 4.5, or 5.0 mm.

In certain embodiments, fabrication of the assay may result in portions of the assay overlapping with each other, as illustrated in FIGS. 10 and 11. As illustrated in FIG. 10, an assay 1000 receives a sample containing an analyte 1002 onto the sample pad 1005, which overlies a PVC baseplate 1001. The sample pad 1005 may overlap with the conjugate pad 1010 that extends from a first end of the sample pad 1005 to an opposite end of an absorbent pad 1040. The sample pad 1005 is raised above the conjugate pad 1010, which may comprise a first molecule 1015 that specifically binds to an antigen in the analyte 1002. In certain embodiments, the conjugate pad 1010 overlaps with the NC membrane 1018, such that the conjugate pad 1010 is raised above the NC membrane 1018. In certain embodiments, the NC membrane 1018 overlaps with the absorbent pad 1040, such that the absorbent pad is raised above the NC membrane 1018. In other certain embodiments, the conjugate pad comprises a biotinylated pad 1110a and a gold-labeled pad 1110b, as illustrated in FIG. 11. The biotinylated pad overlaps 1110a with the gold-labeled pad 1110b, such that the biotinylated pad 1110a is raised above the gold-labeled pad 1110b. In yet other certain embodiments, the gold-labeled pad 1110b overlaps with the NC membrane 1118, such that the gold-labeled pad 1110b is raised above the NC membrane 1118.

It is preferred to read the results of all analytes at the same time point, for instance, after 15-30 minutes. Alternatively, the results can be read consecutively.

The described samples are PDEs. In an embodiment, the PDE is used directly after draining to detect one or more markers of infection. In a preferred embodiment, the PDE is first concentrated and/or filtered, and subsequently used to detect one or more markers of infection.

The shape of the device has no limit so long as the individual channels are maintained.

Although a lateral flow immunoassay is preferred, other techniques such as quantitative immunofluorescence, dry chemistry, etc. can be adapted for use herein.

Methods: In certain embodiments, after a patient has presented in the clinic with a cloudy PDE and/or other symptoms of infection, the practitioner can quickly use the device before sending samples for WBC count and culture. The practitioner can then interpret the results and provide to the nephrologist or supervisor the test results, symptoms, history, etc., along with a possible antibiotics prescription(s).

Alternatively, the device can be used at home by patients. The patient should inform the clinician(s) of the results immediately and follow the suggested instructions for treatment, e.g., orally taken antibiotics, or adding antibiotics directly into fresh dialysis solution for the next exchange. This may happen, for example, outside of clinic hours or for those patients who live in a rural area, without having to wait to present at clinic before implementing a necessary therapeutic regimen.

In certain embodiments, a reference strip, such as depicted in FIG. 8, provides a mechanism for determining whether H-NGAL and/or inflammatory markers, such as IL-6 proteins, are present in the PDE at greater than or equal to a cut-off value for each protein. The cutoff value for H-NGAL may be greater than or equal to about 10, 20, 30, 40, 50, 60, 70, 80, 90, 100, 200, 300, 400, 500, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000 pg/mL. In certain embodiments, the cutoff value for H-NGAL is greater than or equal to 500 pg/mL. In certain embodiments, IL-6 may be used as the inflammatory marker indicative of an immune response that has launched in the peritoneum, and the cut-off values for IL-6 may be 50, 60, 70, 80, 90, 100, 150, 200, 250, 300, 350, 400, 450, 500, 550, 600, 650, 700, 750, 800, 850, 900, 950, 1000 pg/mL. In certain embodiments, the cut-off value for IL may be 200 pg/mL.

In certain embodiments, a method of treating peritonitis or of recognizing a risk of peritonitis in a subject comprises: detecting H-NGAL and IL-6 in a PDE of a patient in need of treatment, comparing a level of H-NGAL and a level of IL-6 in the PDE against a cut-off value for H-NGAL and a cut-off value for IL-6; and administering at least one antibiotic if the level of H-NGAL is greater than the cut-off value for H-NGAL and if the value of IL-6 is greater than the cut-off value for IL-6.

The at least one antibiotic may be selected from a group of single-agent antibiotics, the group comprising: ertapenem (INVANZ™), cefoxitin, doripenem (DORIBAX™), imipenem/cilastatin (PRIMAXIN™), meropenem (MERREM™), moxifloxacin (AVELOX™), piperacillin/tazobactam (ZOSYN™), ticarcillin/clavulanate (TIMENTIN™) and tigecycline (TYGACIL™). In one embodiment, the combination therapy antibiotics include cefepime (MAXIPIME™), cefotaxime (CLAFORAN™), ceftazidime (FORTAZ™) or ceftriaxone (ROCEPHIN™), together with metronidazole (FLAGYL™); or cefazolin, cefotaxime, ceftriaxone, ciprofloxacin (CIPROT™), levofloxacin (LEVAQUIN™), together with metronidazole; or cefepime, ceftazidime, ciprofloxacin, levofloxacin, together with metronidazole; or gentamicin or tobramycin together with clindamycin (CLEOCINT™) or metronidazole (with or without ampicillin).

In certain embodiments, a healthcare provider or clinician may diagnose a peritoneal dialysis subject by interacting the PDE of the peritoneal dialysis subject with the assay kit. The step of interacting the PDE with the assay involves a kit comprising at least one first binding molecule that binds specifically to NGAL and at least one second binding molecule that specifically binds H-NGAL and not a monomer of NGAL or a heterodimer of NGAL. In certain embodiments, the first binding molecule, the second binding molecule, or both are conjugated with a label (such as a reporter or indicator) that enables detecting H-NGAL, and thus allowing for a diagnosis for peritonitis.

In certain embodiments, the device is utilized as part of a method for diagnosing, preferably quick diagnosis of peritonitis or a risk of peritonitis in a peritoneal dialysis subject comprising: detecting H-NGAL protein in peritoneal dialysis effluent of the subject, where H-NGAL protein is greater than or equal to a cutoff value, indicating that the subject has peritonitis or is at a risk of peritonitis; optionally, the method further comprises detecting IL-6 protein in the peritoneal dialysis effluent. If IL-6 protein is greater than or equal to a cutoff value, then the subject has peritonitis or is at a risk of peritonitis.

In certain embodiments, the method for diagnosing peritonitis involves detecting the presence of H-NGAL protein, the detection step comprising: (a) detecting H-NGAL protein in the PDE; or (b) detecting total NGALs in the PDE, including monomer NGAL, H-NGAL and NGAL heterodimer forms, and detecting the monomer and heterodimer forms, so as to determine the amount of H-NGAL. A cut-off value of 500 pg/mL for H-NGAL may be used, and a cut-off value of 200 pg/mL for IL-6 may be used. In certain embodiments, the method of diagnosis comprises obtaining a PDE sample, contacting a first molecule (e.g., an antibody) that specifically binds to H-NGAL protein with the sample, optionally the first molecule is immobilized on a solid support, and detecting a complex formed by the complexation of the first molecule, H-NGAL, and the second molecule.

In certain other embodiments, a healthcare provider or clinician may obtain a PDE sample and use an assay for detecting peritonitis. The assay allows for the healthcare provider or clinician to introduce the PDE sample, where the PDE sample contacts a first molecule, such as an antibody, that specifically binds to H-NGAL protein within the PDE sample. Optionally, the first molecule is immobilized on a solid support. A second molecule (e.g., a first antibody) that specifically binds to H-NGAL protein, optionally labeled, wherein the second molecule and the first molecule bind to different epitopes on H-NGAL protein. Optionally, a substance that specifically binds to the second molecule, such as a second antibody against the second molecule, may be added. The substance may be optionally labeled. The healthcare provider or clinician may detect a formed complex of the first molecule (the first antibody), H-NGAL, and the second molecule (the second antibody).

In certain other embodiments, the healthcare provider or clinician obtains a PDE sample, wherein the PDE sample contacts a first antibody molecule that specifically binds to H-NGAL protein within the PDE sample. The first antibody molecule is labeled with biotin (i.e., biotinylated). A second antibody molecule that specifically binds to H-NGAL protein may be added, wherein the second antibody molecule is labeled with colloidal gold, and the second antibody molecule and the first antibody molecule are different antibodies that specifically bind to different epitopes on H-NGAL protein. Streptavidin may be added. The streptavidin may be optionally immobilized on a solid support. Streptavidin allows for the detection of the complex formed by the complexation of the biotinylated first antibody, H-NGAL, the gold-labeled second antibody.

Also described herein is a method for treating peritonitis in a peritoneal dialysis subject, the method comprising: (a) detecting H-NGAL protein in a peritoneal dialysis effluent of the subject; (b) determining a level of H-NGAL; (c) comparing the level of H-NGAL against a cutoff value; and (d) administering an antibiotic therapy if the level of H-NGAL exceeds the cutoff value. In certain embodiments, the cut-off value for H-NGAL may be 500 pg/mL. In certain embodiments, administering an antibiotic therapy comprises administering a drug selected from the group consisting of ertapenem, cefoxitin, doripenem, imipenem, cilastatin, meropenem, moxifloxacin, piperacillin, tazobactam, ticarcillin, clavulanate and tigecycline; or cefepime, cefotaxime, ceftazidime or ceftriaxone, together with metronidazole; or cefazolin, cefotaxime, ceftriaxone, ciprofloxacin, levofloxacin, together with metronidazole; or cefepime, ceftazidime, ciprofloxacin, levofloxacin, together with metronidazole; or gentamicin or tobramycin together with clindamycin or metronidazole.

In certain embodiments, provided is a strip for diagnosis, especially quick diagnosis of peritonitis or a risk of peritonitis in a peritoneal dialysis patient, comprising a solid support having a first end and a second end, and from the first end, continuously comprising: a sample pad, a conjugate pad, a nitrocellulose membrane (comprising a test line and a control line) and optionally an absorbent pad (such as shown in FIG. 11), wherein the conjugate pad comprises a first molecule, optionally labeled, that specifically binds to H-NGAL protein; the test line comprises an immobilized substance that detects a complex formed by H-NGAL protein and the first molecule; and the control line comprises an immobilized substance that specifically binds to the first molecule. In a further embodiment, in the strip, the conjugate pad further comprises a third molecule, optionally labeled, that specifically binds to IL-6 protein, and the nitrocellulose membrane further comprises another test line comprising a substance that detects a complex formed by IL-6 protein and the third molecule, and the control line may comprise a substance that binds to the first molecule and/or the third molecule.

Alternatively, the device can be used by clinician(s) or the patient to determine whether the prescribed treatment has been effective.

In certain embodiments, a healthcare provider or clinician may use the assay kit to diagnose an infection in a subject. The healthcare provider or clinician may interact a sample, such as a PDE, from a subject with assays from the assay kit, where the assay kit comprises a first binding molecule that specifically binds to NGAL, a second binding molecule that specifically binds to H-NGAL (and not the NGAL monomer or NGAL heterodimer), wherein the first and second binding molecules are conjugated with a label such as a reporter or indicator that allows for the clinician to detect the presence of H-NGAL in the PDE, and thus enables a diagnosis of an infection in the subject. In other certain embodiments, the healthcare provider or clinician may use the kit to diagnose an infection by detecting the presence of a pathogen. The kit comprises a third binding molecule on a solid phase that specifically binds to a marker indicative of a pathogen and a fourth binding molecule that specifically binds to the marker indicative of a pathogen in the sample. In some embodiments, the healthcare provider or clinician may detect a pathogen such as a bacterium (i.e., a gram-negative bacterium, a gram-positive bacterium) and/or a fungus. Based on the assay kit results, the healthcare provider or clinician may further treat the subject by administering the appropriate antibiotic to the subject to treat the infection caused by the pathogen.

In certain embodiments, the device is utilized as part of an integrated care management protocol. In certain embodiments, results determined by the device are processed by one or more portable electronic devices (e.g., smartphone app, computer, tablet, etc.) and may be associated with a digital application. The results may in turn be integrated via a cloud or network-based communication path to a remote clinician, laboratory, or other health professional for further evaluation and instruction. Such a remote professional may then direct appropriate clinical action by the patient or on-site professional, such as directing immediate administration of an appropriate antibiotic or other therapy, direct the patient to come to the clinic, or otherwise indicate appropriate care. In some embodiments, a remote professional can send an electronic code to open a medicine lockbox, for example, in the patient's home, with the appropriate medication.

In certain embodiments, a healthcare provider or clinician may use the assay kit for diagnosing peritonitis based on the detection of NGAL. In some embodiments, H-NGAL is present in the PDE of a peritoneal dialysis subject at a sufficient concentration to indicate peritonitis in the peritoneal dialysis subject based on a cut-off value of H-NGAL. In other embodiments, the method involves using an assay that is further equipped to detect the presence of IL-6 in the PDE of the peritoneal dialysis patient, wherein a concentration of IL-6 equal to or greater than a cut-off value indicates that the peritoneal dialysis subject has peritonitis or is at risk of developing peritonitis.

An advantage of such protocols is that they can be performed remotely, as by a patient in the home or at a remote clinic, and they can exploit the point of care testing device to rapidly identify a causative agent of infection and rapidly institute suitable treatment (e.g., specific antibiotics). In other cases, these protocols can be performed in a clinic with little additional training required of the practitioner, but nevertheless reducing time to appropriate treatment over conventional protocols.

The invention is further described with the aid of the following illustrative Examples.

### EXAMPLES

### Example I

Preliminary detection of peritonitis in PDE from patients using an antibody pair that is specific for the NGAL homodimer.

The assay was made with the following antibody components incorporated into a paper-based (e.g., nitrocellulose) LFD: a capture antibody, a detection antibody, and a control antibody. The capture antibody was Anti-NGAL (Cat. #4NG7-N457). The detection antibody was Anti-NGAL (conjugated) (Cat. #4NG7-N316), and a control line was, for example, Goat-anti-mouse antibody (ShangDong ShuoJing).

87 PDE samples were tested using Anti-NGAL homodimers in a LFT. Of the 87 PDE samples, 9 PDE samples were collected from patients with peritonitis, and 78 samples were collected from patients without peritonitis. The results of the test demonstrated that the test showed specificity (100%; i.e., no false positives) and sensitivity (66.7%) for the H-NGAL.

### Example II: Manufacturing Strips for a LFA for Detecting Peritonitis

### 1. Materials

The assay comprised a line strip having a first and second end. Between the first and second ends were a sample section, a conjugated section, a nitrocellulose (NC) section, and an absorbent pad. The nitrocellulose section comprised a test line and a control line. Goat-anti-rabbit IgG (Shandong Shuojing Biological Co., Ltd (Cat. No.: 1303)) was immobilized over the control line. Antibodies specific for NGAL, a homodimer of NGAL (H-NGAL), or both were immobilized over the test line. Antibodies specific for H-NGAL were purchased from Hytest Biotechnology (Shanghai) Co., Ltd. (Cat. No.: N316, N457). Antibodies specific for NGAL were purchased from NanJing Genscript (Cat. No.: 4C10A7, 5A9D12).

The conjugated section comprised biotin, which was purchased from Thermo Fisher Scientific (Cat. No.: 21312). The nitrocellulose section was also configured to detect the presence of interleukin-6 (IL-6). Thus, the nitrocellulose (NC) section had antibodies for detecting IL-6, the antibodies were purchased from Novoprotein (Cat. No.: DA011, DA 012).

### 2. Strip Preparation

2.1 Sample Pad. 50 mL of glass fiber treatment solution (adding 24 g tris(hydroxymethyl)aminomethane, 50 g sucrose, 5 g trehalose, 15 g casein, 5 g polyvinylpyrrolidone, 3.5 g anhydrous sodium carbonate and 1 g NaN₃ in order, and dissolving completely by stirring; adding 2 mL of TRITON® X-100 and mixing completely by stirring; adding an appropriate amount of hydrochloric acid to adjust pH to 8.5; and placing at room temperature for use) was poured into a stainless steel dish, and a piece of glass fiber (Ahlstrom, Cat. No.: 8964) was put therein, completely saturated and then taken out. The glass fiber was put on a grid, and fully dried at a temperature of 30-40° C. at an ambient humidity of <20% for 12 hours to obtain a full piece of sample pad and cut according to requirement of product.

2.2 Coating and drying of the nitrocellulose membrane. Control line: fixed spraying amount (1 μL/cm), the nitrocellulose membrane was coated with 1.5 mg/mL of the goat anti-rabbit IgG antibody. Test line: fixed spraying amount (1 μL/cm), the nitrocellulose membrane was coated with 1.0 mg/mL of streptavidin. The control line and the test line were separated by about 5 mm. Drying process parameters: temperature of 30-40° C., dried for 2 hours.

2.3 Gold-labeled pad. 10 μL of K₂CO₃ solution at 1% mass concentration and 20 μg of an antibody (H-NGAL antibody (Cat. No: N316); IL-6 antibody (Cat. No: DA011); and NGAL antibody (Cat. No: 4C10A7)) were added into 1 mL of colloidal gold solution (four ten thousandths of nano-gold, particle size 35 nm). The gold-labeled antibody was concentrated by centrifugation at 10:1, and the concentrated gold-labeled antibody solution was diluted at a ratio of 10%-12%; then it was uniformly immobilized on the glass fiber and dried at a temperature of 30-40° C. for 12 hours. The gold-labeled antibody reaction membrane was cut into a width of 5 mm for use.

2.4 Biotinylated pad. The coating antibodies (H-NGAL antibody (Cat. No: N457); IL-6 antibody (Cat. No: DA012); NGAL antibody (Cat. No: 5A9D12)) were biotinylated with biotin at a molar ratio of 1:20. After reacting at 2-8° C. in the dark for 2 hours, unreacted biotin was removed via dialysis. The biotinylated antibody solution was diluted at a ratio of 0.35%-0.5%, evenly immobilized on the glass fiber, and dried at a temperature of 30-40° C. for 12 hours, and then the dried biotin antibody reaction membrane was cut into a width of 5 mm for use.

2.5 Assembly. The following were placed on a baseplate: the prepared nitrocellulose membrane, the upper absorbent paper (partially overlapped with the nitrocellulose membrane (about 1.0-1.5 mm) and above it), and the gold-labeled pad (partially overlapped with the nitrocellulose membrane (about 1.0-1.5 mm) and above it), followed by the biotinylated pad (partially overlapped with the gold-labeled pad (about 1.0-1.5 mm) and above it), and finally the sample pad obtained in 2.1 (partially overlapped with the biotinylated pad (by about 1.0-1.5 mm) and above it). The prepared strips from one end to the other comprised: the upper absorbent paper, the nitrocellulose membrane (sequentially the control line and the test line), the gold-labeled pad, the biotinylated pad, and the sample pad (with sample loading wells thereon). The assembled test strip is shown in FIG. 9.

Finally, handle stickers, MAX tape (strip type) and white stickers (card type, pen type) were attached, cut into the required width, and stored at 4° C.-30° C. in the dark.

### 3. Preparation of Standards of NGAL, IL-6 and H-NGAL

NGAL (recombinant neutrophil gelatinase-associated lipocalin antigen (monomer)): Shandong Shuojing Biological Co., Ltd., Cat. No.: 190703.

H-NGAL (recombinant neutrophil gelatinase-associated lipocalin antigen (Homodimer)): Nanjing AIDPRO Biotech Technology Co., Ltd., Cat. No.: AEP0061.

IL-6 (interleukin 6): NIBSC, Cat. No.: 89-548.

The respective standards of NGAL, IL-6 and H-NGAL were diluted in series with negative peritoneal dialysis effluent samples to obtain NGAL, IL-6 and H-NGAL solutions of various concentrations. The solutions of different concentrations were assayed by strips having different cutoff values, and each concentration was repeated at least 3 times.

### Example III: Diagnosis and Assay of Peritonitis

According to ISPD guidelines (ISPD Guidelines/Recommendations, ISPD Peritonitis Recommendations: 2016 Update On Prevention And Treatment, Peritoneal Dialysis International, Vol. 36, pp. 481-508), peritonitis in patients is diagnosed when at least 2 of the following are present: (1) clinical features consistent with peritonitis, i.e., abdominal pain and/or cloudy dialysis effluent; (2) dialysis effluent white cell count >100/pL or >0.1×10⁹/L (after a dwell time of at least 2 hours); and (3) positive dialysis effluent culture.

Peritonitis in peritoneal dialysis patients were diagnosed according to the above criteria. Peritoneal dialysis includes continuous ambulatory peritoneal dialysis (CAPD) and automatic peritoneal dialysis (APD). Following the doctor's discretion and prescription, the subjects were subject to peritoneal dialysis by using the volume of peritoneal dialysis solution as prescribed every several hours. As described above, peritoneal dialysis was performed according to the ISPD guidelines (ISPD GUIDELINES/RECOMMENDATIONS, Peritoneal Dialysis International, Vol. 36, pp. 481-508).

### 4. Detection Method

4.1 Obtained samples of PDE from peritoneal dialysis patients.

4.2 Added 70-80 μL (2 drops) of the PDE sample to the sample loading wells of the strips as prepared in Example II, and allowed to react for 10-15 minutes.

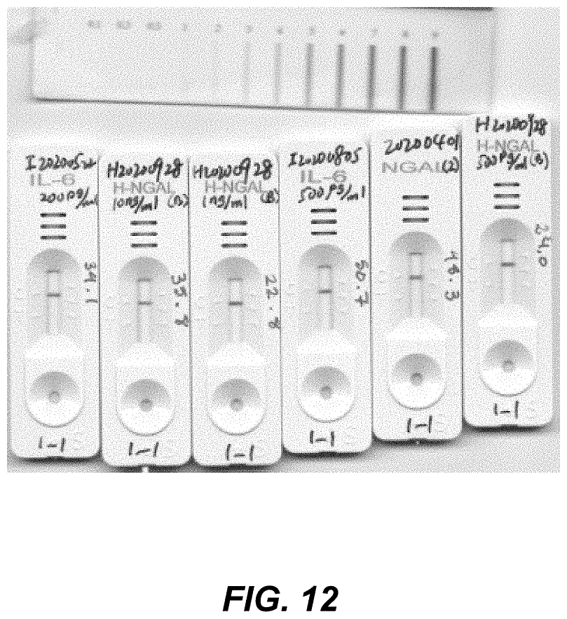

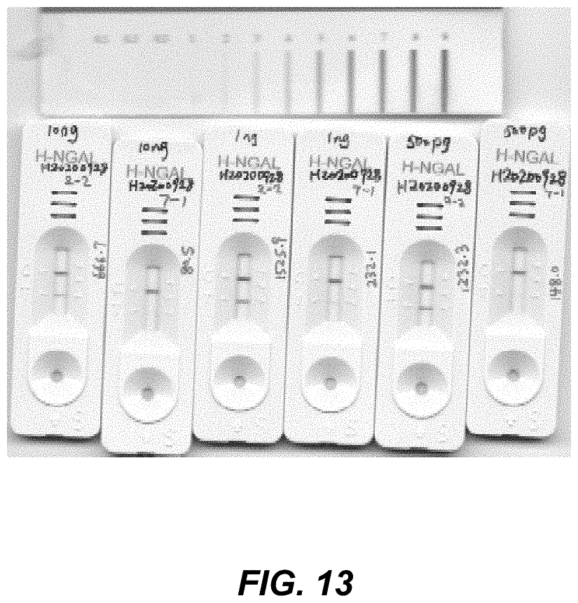

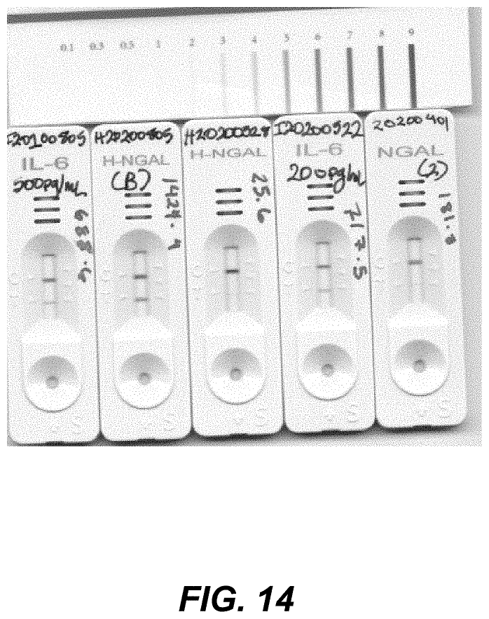

4.3 Compared the strips with the reference colorimetric card (as shown in FIG. 8), and reading the coloration results, where a coloration intensity of >0.5 is positive. Negative result=positive control line+negative test line; positive result=positive control line +positive test line. Some of the results are shown in FIGS. 12-14.

### 5. Detection and Results

5.1 IL-6 and NGAL (total NGALs, including the monomer, homodimer and heterodimer forms) proteins were detected blindly in the peritoneal dialysis effluents of peritoneal dialysis patients suffering from peritonitis and clinically diagnosed to be free of peritonitis. The detection results are summarized in Table 1 below.

For example, “NGAL (80 ng/mL)” corresponds to a cut-off value of 80 ng/mL for H-NGAL in a particular assay unless indicated otherwise. By similar example, “IL-6 (1000 pg/mL)” denotes a cut-off value of 1000 pg/mL for IL-6 for a particular assay unless indicated otherwise. For assays that detected both NGAL and IL-6, those assays are designated with an “AND” in the first column. “Single” indicates that only H-NGAL or only IL-6, but not both, were tested for in a particular assay as described above.

TABLE 1
Detection of IL-6 and/or NGAL

| Number | | | Specificity | | | | Sensitivity | | | |
|---|---|---|---|---|---|---|---|---|---|---|
| of | | | | | Number | | | | Number | |
| Proteins | Target Protein and | Target Protein | False | | of | | False | | of | |
| Detected | cutoff value | and cutoff value | positive | Negative | samples | Specificity | negative | Positive | samples | Sensitivity |
| Single | NGAL (100 ng/mL) | / | 15 | 36 | 51 | 70.6% | 4 | 46 | 50 | 92.0% |
| Single | NGAL (80 ng/mL) | / | 12 | 20 | 32 | 62.5% | 3 | 47 | 50 | 94.0% |
| Single | NGAL (120 ng/mL) | / | 4 | 28 | 32 | 87.5% | 5 | 45 | 50 | 90.0% |
| Single | IL6 (200 pg/mL) | / | 2 | 49 | 51 | 96.1% | 0 | 50 | 50 | 100.0% |
| Single | IL6 (500 pg/mL) | / | 1 | 31 | 32 | 96.9% | 1 | 49 | 50 | 98.0% |
| Single | IL6 (1000 pg/mL) | / | 1 | 31 | 32 | 96.9% | 3 | 47 | 50 | 94.0% |
| AND | NGAL (100 ng/mL) | IL-6 (200 pg/mL) | 1 | 50 | 51 | 98.0% | 4 | 46 | 50 | 92.0% |
| AND | NGAL (100 ng/mL) | IL-6 (500 pg/mL) | 1 | 31 | 32 | 96.9% | 5 | 45 | 50 | 90.0% |
| AND | NGAL (100 ng/mL) | IL-6 (1000 pg/mL) | 1 | 31 | 32 | 96.9% | 6 | 44 | 50 | 88.0% |
| AND | NGAL (80 ng/mL) | IL-6 (200 pg/mL) | 2 | 30 | 32 | 93.8% | 3 | 47 | 50 | 94.0% |
| AND | NGAL (80 ng/mL) | IL-6 (500 pg/mL) | 1 | 31 | 32 | 96.9% | 4 | 46 | 50 | 92.0% |
| AND | NGAL (80 ng/mL) | IL-6 (1000 pg/mL) | 1 | 31 | 32 | 96.9% | 5 | 45 | 50 | 90.0% |
| AND | NGAL (120 ng/mL) | IL-6 (200 pg/mL) | 1 | 31 | 32 | 96.9% | 5 | 45 | 50 | 90.0% |
| AND | NGAL (120 ng/mL) | IL-6 (500 pg/mL) | 1 | 31 | 32 | 96.9% | 6 | 44 | 50 | 88.0% |
| AND | NGAL (120 ng/mL) | IL-6 (1000 pg/mL) | 1 | 31 | 32 | 96.9% | 7 | 43 | 50 | 86.0% |

Note: “AND” means that the result was considered as positive, i.e., peritonitis, when both NGAL and IL-6 were positive. NGAL means detection of total NGAL proteins, including the monomer, homodimer and heterodimer forms.

According to the results in Table 1, for detecting NGAL, using a cut-off value of 120 ng/mL showed better specificity (87.5%) and sensitivity (90.0%) than other cut-off values; for detecting IL-6, using a cut-off value of 200 pg/mL showed a sensitivity of 100% and a specificity of 96.1%, which is slightly lower than the specificities based on lower cut-off values. However, both specificity and sensitivity for detecting NGAL were lower than when detecting IL-6.

5.2 H-NGAL, IL-6 and NGAL (total NGALs, including the monomer, homodimer and heterodimer forms) were detected blindly in dozens of peritoneal dialysis effluents of peritoneal dialysis patients suffering from peritonitis and clinically diagnosed to be free of peritonitis. The detection results are summarized in Table 2 below.

Table 2 shows detection of H-NGAL (500 pg/mL) (sensitivity of 98.18%, specificity of 96.08%) and detection (“AND”) of both H-NGAL (500 pg/mL) and IL-6 (200 pg/mL) (sensitivity of 98.18%, specificity of 97.87%) showed high sensitivity and specificity.

In a later stage, to further validate the result above, more peritoneal dialysis effluent samples were tested, and the testing results were appended to the results in Table 2 to give Table 3 below.

TABLE 2
Detection of IL-6 and/or NGAL/H-NGAL

| Number | | | Specificity | | | | Sensitivity | | | |
|---|---|---|---|---|---|---|---|---|---|---|
| of | | | | | Number | | | | Number | |
| Proteins | Target Protein and | Target Protein | False | | of | | False | | of | |
| Detected | cutoff value | and cutoff value | positive | Negative | samples | Specificity | negative | Positive | samples | Sensitivity |
| Single | NGAL (120 ng/mL) | / | 7 | 25 | 32 | 78.13% | 8 | 46 | 54 | 85.19% |
| Single | IL-6 (200 pg/mL) | / | 5 | 42 | 47 | 89.36% | 1 | 54 | 55 | 98.18% |
| Single | H-NGAL (500 pg/mL) | / | 2 | 49 | 51 | 96.08% | 1 | 54 | 55 | 98.18% |
| Single | H-NGAL (1 ng/mL) | / | 2 | 49 | 51 | 96.08% | 2 | 53 | 55 | 96.36% |
| Single | H-NGAL (10 ng/mL) | / | 1 | 50 | 51 | 98.04% | 3 | 52 | 55 | 94.55% |
| AND | NGAL (120 ng/mL) | IL-6 (200 pg/mL) | 4 | 28 | 32 | 87.50% | 8 | 46 | 54 | 85.19% |
| AND | H-NGAL (500 pg/mL) | IL-6 (200 pg/mL) | 1 | 46 | 47 | 97.87% | 1 | 54 | 55 | 98.18% |
| AND | H-NGAL (1 ng/mL) | IL-6 (200 pg/mL) | 1 | 46 | 47 | 97.87% | 2 | 53 | 55 | 96.36% |
| AND | H-NGAL (10 ng/mL) | IL-6 (200 pg/mL) | 1 | 46 | 47 | 97.87% | 3 | 52 | 55 | 94.55% |

Note: “AND” means that the result was positive when both NGAL/H-NGAL and IL-6 were positive. NGAL: detecting total NGALs, including monomer (synthesized by renal tubular epithelial cells), homodimer (synthesized by leukocytes/neutrophils) and heterodimer (associated with MMP9)

TABLE 3
Detection of IL-6 and/or NGAL/H-NGAL with more samples of PD effluents tested

| | | | Specificity | | | | Sensitivity | | | |
|---|---|---|---|---|---|---|---|---|---|---|
| | | | Criterial: Specificity. >= 94.5% | | | | Criterial: Sensitivity. >= 98.5% | | | |
| | | | False | | | | False | | | |
| Number of Proteins Detected | | | positive | Negative | Total | Specificity | Negative | Positive | Total | Sensitivity |
| Single | NGAL | 120 ng/mL | 11 | 64 | 75 | 85.33% | 9 | 48 | 57 | 84.21% |
| Single | IL6 | 200 pg/mL | 7 | 83 | 90 | 92.22% | 1 | 57 | 58 | 98.28% |
| Single | H-NGAL | 500 pg/mL | 2 | 92 | 94 | 97.87% | 1 | 57 | 58 | 98.28% |
| Single | H-NGAL | 1 ng/mL | 2 | 92 | 94 | 97.87% | 2 | 56 | 58 | 96.55% |
| Single | H-NGAL | 10 ng/mL | 1 | 93 | 94 | 98.93% | 3 | 55 | 58 | 94.83% |
| AND | NGAL (120 ng/mL) | IL-6 (200 pg/mL) | 5 | 70 | 75 | 93.33% | 9 | 48 | 57 | 84.21% |
| AND | H-NGAL (500 pg/mL) | IL-6 (200 pg/mL) | 1 | 89 | 90 | 98.89% | 1 | 57 | 58 | 98.28% |
| AND | NGAL (1 ng/mL) | IL-6 (200 pg/mL) | 1 | 89 | 90 | 98.89% | 2 | 56 | 58 | 96.55% |
| AND | NGAL (10 ng/mL) | IL-6 (200 pg/mL) | 1 | 89 | 90 | 98.89% | 3 | 55 | 58 | 94.83% |

Note: “AND” means that the result was positive when both NGAL/H-NGAL and IL-6 were positive. NGAL: detecting total NGALs, including monomer (synthesized by renal tubular epithelial cells), homodimer (synthesized by leukocytes/neutrophils) and heterodimer (associated with MMP9). “Total” means total number of samples.

Table 3 shows detection of H-NGAL (500 pg/mL) (sensitivity of 98.28%, specificity of 97.87%) and detection (“AND”) of both H-NGAL (500 pg/mL) and IL-6 (200 pg/mL) (sensitivity of 98.28%, specificity of 98.89%) showed high sensitivity and specificity.

### 6. Conclusions

a) The single IL-6 assay using a cut-off value of 200 pg/mL demonstrated the best sensitivity and specificity at 100% and 96.1%, respectively, of the tested cut-off values for IL-6 (see Table 1).

b) According to the results in Table 1 above, the cut-off values for total NGAL, IL-6, and H-NGAL were determined to be 120 ng/mL, 200 pg/mL, and 500 pg/mL, respectively.

c) The results in Tables 2 and 3 show that detection of H-NGAL using anti-H-NGAL antibodies showed higher specificity and sensitivity than detection of total NGAL using anti-NGAL antibodies. Detection of H-NGAL showed higher specificity than detection of IL-6.

d) Detection of H-NGAL (500 pg/mL) (sensitivity of 98.28%, specificity of 97.87%) and detection of both H-NGAL (500 pg/mL) and IL-6 (200 pg/mL) (“AND”) (sensitivity of 98.28%, specificity of 98.89%) showed high sensitivity and specificity.

### REFERENCES

(the contents of which are incorporated herein by this reference)
Gong Pin et al., “Research progress of colloid gold immunochromatographic test strip technology and its application in food safety testing,” Food Industry Science and Technology, 2019, 40(13): 358-364, Article ID: 1002-0306(2019)13-0358-07.
International Society for Peritoneal Dialysis (ISPD GUIDELINES/RECOMMENDATIONS, Peritoneal Dialysis International, Vol. 36, pp. 481-508).
Klein et al. “Murine anti-interleukin 6 monoclonal antibody therapy for a patient with plasma cell leukemia,” Blood 78, 1198-1204 (1991).
Linjun Cai et al., “Assays of urine levels of HNL/NGAL in patients undergoing cardiac surgery and the impact of antibody configuration on their clinical performances,” Clinica Chimica Acta 403 (2009) 121-125, DOI: 10.1016/j.cca.2009.01.030.
Linjun Cai et al., “The origin of multiple molecular forms in urine of HNL/NAGL,” Clin. J. Am. Soc. Nephrol. 5:2229-2235, 2010, DOI: 10.2215/CJN.00980110).
“Neutrophil gelatinase-associated lipocalin (NGAL),” TechNotes, p. 2 (January 2020 by HyTest Ltd.).
Ronco C, Dell′Aquila R, Rodighiero MP (eds): “Peritoneal Dialysis: A Clinical Update,” Contrib. Nephrol., Basel, Karger, 2006, vol. 150, pp 187-194.
Song M, Kellum JA. Interleukin-6. Crit Care Med 2005; 33 (Suppl. 12): 463-465.
Yan Lingzhi, “Research progress of immunochromatographic test strip technology in the field of food safety testing,” Food Industry Science and Technology, DOI: 10.13386/j.issn1002-0306.2020110079.
EP 0430193 to Biotest Pharma GmbH (Jun. 5, 1991).
US 20050250141A1 to Lambert et al. (Nov. 10, 2005).
PCT International Publication WO2018/060708.
PCT International Publication WO 20200249228 (Aug. 6, 2020).
U.S. Pat. No. 6,924,153 B1 to Boehringer et al. (Aug. 2, 2005).
U.S. Pat. No. 7,785,899 B2 to Saul et al. (Feb. 18, 2005).
U.S. Pat. No. 7,893,219 to Cassone et al. (Feb. 22, 2011).
U.S. Pat. No. 8,128,871 B2 to Petruno et al. (Mar. 6, 2012).

What is claimed is:

1. An assay kit for diagnosing an infection in a subject, the assay kit comprising:
at least one first binding molecule that specifically binds neutrophil gelatinase-associated lipocalin (NGAL) in a peritoneal dialysis effluent (PDE) of a subject, and
at least one second binding molecule that specifically binds NGAL homodimer (H-NGAL), but does not bind a monomer of NGAL or a heterodimer of NGAL in the PDE,
wherein the first binding molecule, the second binding molecule, or both the first and second binding molecule are conjugated with a label.

2. The assay kit of claim 1, the assay kit further comprising:
at least one third binding molecule on a solid phase that binds a marker indicative of a presence of a pathogen, and
at least one fourth binding molecule that specifically binds to the marker indicative of a presence of a pathogen in the PDE.

3. The assay kit of claim 1, wherein either the first or the second binding molecule is on a solid phase of a lateral flow device.

4. The assay kit of claim 2, wherein the pathogen is selected from the group consisting of a bacterium and a fungus.

5. An assay kit comprising:
a binding molecule that, within a context of the assay kit, specifically binds to neutrophil gelatinase-associated lipocalin (NGAL) homodimer (H-NGAL), but not to a monomer or a heterodimer of NGAL;
a binding molecule that specifically binds an antigen indicative of a presence of a gram-positive bacteria; and
a binding molecule that specifically binds an antigen indicative of the presence of gram-negative bacteria.

6. The assay kit of claim 5, wherein the antigen indicative of the presence of a gram-positive bacterium is lipoteichoic acid (LTA).

7. The assay kit of claim 5, wherein the antigen indicative of the presence of a gram-negative bacterium is lipopolysaccharide (LPS).

8. A method of diagnosing peritonitis in a subject who is a peritoneal dialysis patient, the method comprising:
detecting in a peritoneal dialysis effluent (PDE) from the subject an antigen indicative of a presence of a microorganism selected from the group consisting of gram-positive bacteria, gram-negative bacteria, fungus, and any combination thereof; and
detecting in the peritoneal dialysis effluent neutrophil gelatinase-associated lipocalin (NGAL) homodimer (H-NGAL);
wherein detecting in a peritoneal dialysis effluent from the subject comprises contacting the H-NGAL with a first molecule that specifically binds to H-NGAL, but wherein the first molecule does not bind to a monomer or a heterodimer of NGAL.

9. The method according to claim 8, wherein the antigen is lipoteichoic acid (LTA).

10. The method according to claim 8, wherein the antigen is lipopolysaccharide (LPS).

11. A method of treating a peritoneal dialysis patient suspected as having peritonitis, the method comprising:
detecting in a peritoneal dialysis effluent (PDE) from the patient at least one antigen indicative of a presence of a microorganism selected from the group consisting of gram-positive bacteria, gram-negative bacteria, fungus, and any combination thereof;
detecting in the peritoneal dialysis effluent neutrophil gelatinase-associated lipocalin (NGAL) homodimer (H-NGAL); and
administering a selected antibiotic to the patient, wherein an appropriate antibiotic is selected based upon results of the detection of particular antigen(s) in the PDE,
wherein an amount of H-NGAL is greater than or equal to a cut-off value of H-NGAL in the PDE.

12. A strip for diagnosing peritonitis, the strip comprising:
a solid support comprising a sample pad, the sample pad configured to receive a sample from a patient, the sample being a peritoneal dialysis effluent (PDE);
a conjugate pad adjacent to the sample pad, wherein the conjugate pad comprises a first molecule that specifically binds to neutrophil gelatinase-associated lipocalin homodimer (H-NGAL), but not to a monomer or heterodimer of NGAL; and
a nitrocellulose membrane, the nitrocellulose membrane comprising a control line and a test line.

13. The strip of claim 12, wherein the test line comprises an immobilized substance that detects a complex of H-NGAL and the first molecule, and the control line comprises an immobilized substance that specifically binds to the first molecule.

14. The strip of claim 12, wherein the conjugate pad comprises a third molecule that specifically binds to interleukin-6 (IL-6).

15. The strip of claim 14, wherein the conjugate pad comprises a fourth molecule that specifically binds to IL-6, wherein the third molecule and the fourth molecule are different.

16. The strip of claim 15, wherein the nitrocellulose membrane further comprises a further test line comprising an immobilized substance that specifically binds to a complex comprising IL-6 and the third molecule, and the control line specifically binds to the third molecule.

17. The strip of claim 12, wherein the conjugate pad comprises biotin, a fluorescent molecule, a luminescent molecule, an enzyme, a molecule with a colloidal gold label, or a combination of any thereof.

18. The strip of claim 12, wherein the first molecule is selected from the group consisting of an antibody, an antigen-binding fragment, a ligand, an aptamer, a polyclonal antibody, a monoclonal antibody, a humanized antibody, a human antibody, a chimeric antibody, and a combination thereof.

19. The strip of claim 12, wherein the conjugate pad comprises a biotinylated pad.

20. The strip of claim 19, wherein the test line comprises streptavidin.

21. A method of diagnosing an infection in a subject, the method comprising:
interacting peritoneal dialysis effluent (PDE) taken from a subject with an assay kit comprising:
at least one first binding molecule that specifically binds neutrophil gelatinase-associated lipocalin (NGAL), and
at least one second binding molecule that specifically binds NGAL homodimer (H-NGAL), but does not bind a monomer of NGAL or a heterodimer of NGAL,
wherein the first binding molecule, the second binding molecule, or both the first and second binding molecule are conjugated with a label; and
detecting a presence of H-NGAL in the PDE with aid of the label so as to determine that the subject is suffering from an infection.

22. The method according to claim 21, wherein the assay kit further comprises:
at least one third binding molecule on a solid phase that binds a marker indicative of the presence of a pathogen;
at least one fourth binding molecule that specifically binds to the marker indicative of the presence of the pathogen in the PDE; and
wherein the method further comprises detecting the presence of the pathogen in the PDE.

23. The method according to claim 22, wherein the pathogen is selected from the group consisting of a bacterium, a gram negative bacterium, a gram positive bacterium, and a fungus; and wherein the method further comprises utilizing the assay kit to characterize the detected pathogen in the PDE as a bacterium, a gram negative bacterium, a gram positive bacterium, or a fungus.

24. The method according to claim 23, further comprising:
treating the subject by administering an antibiotic to the subject to treat the infection caused by the detected pathogen.

25. A molecule that specifically binds to neutrophil gelatinase-associated lipocalin (NGAL) homodimer (H-NGAL) protein for diagnosing peritonitis in a peritoneal dialysis subject by detecting H-NGAL protein in a peritoneal dialysis effluent of the peritoneal dialysis subject.

26. A method for diagnosing peritonitis, the method comprising:
detecting neutrophil gelatinase-associated lipocalin (NGAL) homodimer (H-NGAL) protein in a peritoneal dialysis effluent (PDE) of a subject, wherein a concentration of H-NGAL in the PDE is greater than or equal to a cut-off value indicates that the subject has peritonitis or is at a risk of peritonitis;
optionally, the method further comprises detecting IL-6 protein in the PDE, wherein a concentration of IL-6 protein is greater than or equal to a cutoff value indicates that the subject has peritonitis or is at a risk of peritonitis.

27. The method of claim 26, wherein detecting H-NGAL comprises:
detecting H-NGAL protein in the peritoneal dialysis effluent (PDE); or
detecting total NGALs in the peritoneal dialysis effluent, including monomer NGAL, H-NGAL and heterodimer NGAL, and detecting the monomer and heterodimer forms of NGAL.

28. The method of claim 26, wherein the cutoff value for H-NGAL is 500 pg/mL and optionally, the cut-off value for IL-6 is 200 pg/mL.

29. The method of claim 26, the method further comprising:
obtaining a peritoneal dialysis effluent (PDE) sample;
contacting a first molecule, the first molecule comprising an antibody, that specifically binds to H-NGAL protein with the PDE sample to form a complex, optionally the first molecule is immobilized on a solid support; and
detecting the complex.

30. The method of claim 26, the method further comprising:
obtaining a peritoneal dialysis effluent (PDE) sample;
contacting a first molecule, the first molecule comprising a first antibody, that specifically binds to H-NGAL protein with the PDE sample, optionally the first molecule is immobilized on a solid support;
adding a second molecule, the second molecule comprising a second antibody, that specifically binds to H-NGAL protein, the first molecule is optionally labeled, wherein the first molecule and the second molecule bind to different epitopes on H-NGAL protein;
optionally, adding a substance that specifically binds to the second molecule, such as an antibody against the second molecule, optionally labeled; and
detecting a complex, the complex comprising the first molecule, the second molecule, and H-NGAL protein.

31. The method of claim 26, comprising:
obtaining a peritoneal dialysis effluent (PDE) sample;
contacting a first antibody molecule that specifically binds to H-NGAL protein with the sample, wherein the first antibody molecule is labeled with biotin to produce a biotinylated first antibody molecule;
adding a second antibody molecule that specifically binds to H-NGAL protein, wherein the second antibody molecule is labeled with colloidal gold to produce a gold-labeled second antibody molecule, and the second antibody molecule and the first antibody molecule are different antibodies that specifically bind to different epitopes on H-NGAL protein;
adding streptavidin, optionally, wherein the streptavidin is immobilized on a solid support; and
detecting a complex, the complex comprising the biotinylated first antibody molecule, H-NGAL, and the gold-labeled second antibody molecule.

32. A method of treating peritonitis in a peritoneal dialysis subject, comprising:
detecting neutrophil gelatinase-associated lipocalin homodimer (H-NGAL) protein in a peritoneal dialysis effluent of a subject;
determining a level of H-NGAL;
comparing the level of H-NGAL against a cutoff value; and
administering an antibiotic therapy if the level of H-NGAL exceeds the cutoff value.

33. The method of claim 32, wherein the cutoff value for H-NGAL is 500 pg/mL.

34. The method of claim 32, wherein the antibiotic therapy is selected from the group consisting of ertapenem, cefoxitin, doripenem, imipenem, cilastatin, meropenem, moxifloxacin, piperacillin, tazobactam, ticarcillin, clavulanate tigecycline; cefepime, cefotaxime, ceftazidime or ceftriaxone, together with metronidazole; or cefazolin, cefotaxime, ceftriaxone, ciprofloxacin, levofloxacin, together with metronidazole; ceftazidime, ciprofloxacin, levofloxacin, together with metronidazole; and gentamicin or tobramycin together with clindamycin or metronidazole.
